(19) United States
(12) Patent Application Publication
(10) Pub. No.: US 2020/0321096 A1
(43) Pub. Date: Oct. 8, 2020

(54) SYSTEMS AND METHODS FOR DRUG-AGNOSTIC PATIENT-SPECIFIC DOSING REGIMENS

(71) Applicant: Diane R. Mould, Fort Myers, FL (US)

(72) Inventor: Diane R. Mould, Fort Myers, FL (US)

(21) Appl. No.: 16/813,366

(22) Filed: Mar. 9, 2020

Related U.S. Application Data
(63) Continuation-in-part of application No. 16/391,950, filed on Apr. 23, 2019.
Application No. 16/391,950 is a continuation-in-part of application No. PCT/US2019/028750, filed on Apr. 23, 2019.
(60) Provisional application No. 62/815,825, filed on Mar. 8, 2019.

(51) Int. Cl. G16H 20/17; G16H 70/40; G16H 10/60; G16H 10/40; G16H 50/20; G16H 50/50; G16H 50/30

(52) U.S. Cl. CPC G16H 20/17; G16H 70/40; G16H 10/60; G16H 50/30; G16H 50/20; G16H 50/50; G16H 10/40

(57) ABSTRACT

The systems and methods described herein determine a patient-specific pharmaceutical dosing regimen for a patient, using a computerized pharmaceutical dosing regimen recommendation system. The systems and methods herein provide for a drug-agnostic model, such that the computerized pharmaceutical dosing regimen recommendation system is configured to evaluate and recommend one or more patient-specific dosing regimens that apply to not only a single drug but a class of drugs. The drug-agnostic model provides greater utility than drug-specific models by accessing a broad range of clinical data gathered for all of the drugs in the class of drugs. Rather than implementing multiple models, where each model corresponds to a single drug in a class, a drug-agnostic model is applicable to all drugs within the class and may accordingly be applied to a wider range of patients than single-drug models and across a range of administration routes.

### CROSS-REFERENCE TO RELATED APPLICATIONS

This application claims the priority to and benefit of U.S. Provisional Patent Application Ser. No. 62/815,825, filed on Mar. 8, 2019, and U.S. patent application Ser. No. 16/391,950, filed on Apr. 23, 2019, published as U.S. Patent Publication No. 2019/0326002 on Oct. 24, 2019, related to International Application No. PCT/US19/28750, filed on Apr. 23, 2019. The entire contents of each of the above-referenced applications are hereby incorporated by reference.

### TECHNICAL FIELD

This disclosure relates generally to patient-specific dosing and treatment recommendations including, without limitation, computerized systems and methods that use medication-specific mathematical models specific to a class of drugs and observed patient-specific responses to treatment, to predict, propose, modify and evaluate suitable medication treatment plans for a specific patient.

### BACKGROUND

A physician's decision to start a patient on a medication-based treatment regimen involves determination of a dosing regimen for the medication to be prescribed. Different dosing regimens are appropriate for different patients having differing patient factors. By way of example, dosing quantities, dosing intervals, treatment duration and other variables may vary across different dosing regimens. For example, a patient with low neutrophil counts may require a delayed dose, a lower dose than a typical patient, or both. Traditionally, a physician prescribes an initial dosing regimen based on the package insert (PI) for a drug and the physician's own personal clinical experience. After an initial period of treatment, a physician may follow-up with the patient to reevaluate the patient and reconsider the initial dosing regimen. The PI sometimes provides quantitative indications for increasing or decreasing a dose, or increasing or decreasing a dosing interval. Based on both the physician's assessment of the patient, his clinical experience, and the PI information, the physician would adjust the dosing regimen on an ad hoc basis. Adjusting the dosing regimen for a patient is largely a trial and error process, informed by physicians' experience and clinical judgment.

A proper dosing regimen may be highly beneficial and therapeutic, while an improper dosing regimen may be ineffective or even deleterious to the patient's health. Further, both under-dosing and overdosing generally results in a loss of time, money and/or other resources, and increases the risk of undesirable outcomes. Computerized dosing recommendation systems may assist medical professionals in providing and assessing dosing regimens. Prior disclosed examples of systems and methods are described in U.S. Pat. No. 10,083,400, filed Oct. 7, 2013, issued Sep. 25, 2018, and U.S. application Ser. No. 15/094,379, filed Apr. 8, 2016, published as U.S. Patent Application Publication No. 2016/0300037 A1 on Oct. 13, 2016, each of which is hereby incorporated by reference in its entirety.

Some known population pharmacokinetic models are developed to describe a particular drug and are specific to not only the drug, but to a specific patient population taking the drug. Such models frequently are specific to a particular route of administration as well. These models are intended to be as specific as possible for both the drug, the route of administration and the patients who are expected to take the drug during the course of treatment of a specific disease, so that the model can be used to simulate other dose regimens, compare the pharmacokinetics of the drug after formulations are changed, and various other applications. This approach of developing a model for a single drug limits its application to that drug and the specific patient population for which data were collected to build the model. Limiting model development and usage to a single drug narrows the amount of information or data available.

### SUMMARY

Accordingly, systems and methods are disclosed herein for determining a patient-specific pharmaceutical dosing regimen for a patient, using a computerized pharmaceutical dosing regimen recommendation system applied to a class or other grouping of drugs. The systems and methods described herein provide population models capable of being used for a class or set of drugs. In particular, the systems and methods herein can be configured to develop patient-specific dosing regimens based on data indicative of the pharmacokinetic (“PK”) parameters and pharmacodynamic (“PD”) parameters of a plurality of drugs. The model for a group of drugs would be able to predict patient responses and optimize future dosing based on, for example, past measurements of physiological parameters from a different drug and/or route of administration, because the model is agnostic to the specific drug but utilizes patient-specific data based on similarities between drugs. It may also be desirable for a model to be applicable to a set of drugs and agnostic of a specific drug, such that information can be learned about an administered drug by comparing measured data to the model-based simulations of other drugs in the group which can then be studied against past results. A model that is applicable to a group of drugs may be more accommodating to a physician who may want to treat a patient using a multitude of drugs or vary certain treatment factors such as route of administration

A dosing regimen (also referred to as a treatment plan) may include a schedule for dosing, one or more dosing amounts, and/or one or more routes of administration. Dosing regimens are not limited to just one drug, but can include multiple drugs, with the same or different routes of administration. A drug (also referred to as a pharmaceutical, medicine, medication, biologic, compound, treatment, therapy, or any other similar term) is a substance which has a physiological effect when introduced into a body. In some implementations, the systems described herein are not specific to a particular drug but instead apply to a class, or subset or grouping of drugs used in a drug-agnostic model. As used herein, the term “drug” may refer to a single drug or a class or set of drugs.

A class of drugs may be a group of drugs larger than one, which exhibit at least one similar pharmacokinetic (PK) and/or pharmacodynamic (PD) behavior, share a common mechanism of action, or a combination thereof. As an example, a set of drugs may treat the same disease or be used for the same indication, examples of which include general inflammatory disease, inflammatory bowel disease (IBD), ulcerative colitis, Crohn's disease, rheumatoid arthritis, ankylosing spondylitis, psoriatic arthritis, psoriasis, asthma, or multiple sclerosis. A set of drugs may have a similar chemical structure. For example, a set of drugs could include monoclonal antibodies (mAbs), anti-inflammatory compounds, corticosteroids, immunomodulators, antibiotics or biologic therapies. A user such as a doctor, clinician, or a user building a drug-agnostic model may define a class of drugs based on specific criteria, and members of that class may be electronically designated in a database as being part of that class. That database is accessible to systems and methods disclosed herein, for use in determining a class-based dosing regimen that could be used for any drug in the class. In some implementations, a dosing regimen output from the model is not specific to a single drug but is generic to the class of drugs, and suitable for any drug in that class. For example, a dosing regimen may include a drug-agnostic unit measurement (e.g., one unit, two units, three units, etc, where a unit corresponds to a specified amount of an active agent) and a time or times for administration.

The development and application of drug-agnostic models allow different utility than a single, drug-specific model. Rather than implementing multiple models, where each model corresponds to a single drug in a class, a drug-agnostic model is applicable to all drugs within the class and may accordingly be applied to a wider range of patients than single-drug models and across a range of administration routes. For example, the models described herein may include a pharmacokinetic drug-agnostic model capable of being used for all biologics used in the treatment of inflammatory diseases. Such a model can be used to propose dose regimens for fully human monoclonal antibodies (mAb), chimeric mAbs, fusion proteins, and mAb fragments (i.e., a range of drugs with differing pharmacokinetic properties but other similarities such as molecular weight and indication) using the same model. The model can be used in a broad patient population, including inflammatory bowel disease, rheumatoid arthritis, psoriatic arthritis, psoriasis, multiple sclerosis, and other such diseases that arise from immune dysregulation. The development and application of drug-agnostic Bayesian models for agents in other broad drug sets (e.g., the aminoglycoside antibiotics, chemotherapeutic agents that cause low white cell counts, etc.) is similarly feasible.

Drugs within the class may be administered through a variety of routes, such as subcutaneously, intravenously, or orally. Drug-agnostic models may account for route of administration by taking the route of administration as a variable input to the system, allowing greater flexibility for the model. Because the drug-agnostic model applies to a set of drugs, rather than only a single drug, the model may retain patient-specific information when a patient is treated with multiple drugs within the set of drugs. For example, the set of drugs may include infliximab, vedolizumab, adalimumab, and other anti-inflammatory biologics. If a patient is treated with one drug (e.g. infliximab), then later treated with another drug (e.g., vedolizumab), the model may retain all patient-specific data (drug concentration measurements, clearance rates, weight measurements, etc.) from the patient's treatment on infliximab when determining an appropriate dosing regimen once the patient is being treated with the new drug. Retaining patient-specific data allows the drug-agnostic model to accurately anticipate the patient's ability to process a drug and thereby provide more suitable, patient-specific dosing regimens when a patient changes drug therapy.

One aspect of the present invention relates to a method for generating a patient-specific medication dosing regimen. As discussed in detail below, the method may be implemented on a system which may be a computer system including a single computer or multiple computers communicating over any network, such as in distributed architecture. At least one processor may be housed in one, some, or all of the computers in the computer system, and may be in communication with at least one electronic database stored on the same computer or on a different computer within the computer system. The system may include a cloud-based computing system operated by the same, related, or unrelated entities.

The method includes receiving inputs into the processor of the system. The inputs may include drug data indicative of a plurality of drugs and associated routes of administration, wherein the drugs in the plurality of drugs are expected to exhibit similar pharmacokinetic (PK) behavior, similar pharmacodynamic (PD) behavior, or both. For example, the drugs may have similar chemical structure or mechanism of action, leading to similar pharmacokinetic (PK) behavior, similar pharmacodynamic (PD) behavior, or both. The inputs may include concentration (for a PK model) or response (for a PD model) data indicative of a concentration or response level of a specific drug of the plurality of drugs in a sample obtained from the patient. The inputs may include a target drug exposure or response level for the patient. The target may be decided by a physician based on the drug data and/or concentration or response. In some implementations, a target may be automatically determined by the system in order to result in a therapeutic response in the patient. The system may evaluate a plurality of targets inputted in order to determine one or more targets that result in a therapeutic response in the patient.

The method includes selecting a mathematical model from a database stored in a memory of the system. The database may be accessible by the processor and may store a plurality of mathematical models. The selected mathematical model may be representative of responses by a plurality of patients to one or more drugs in the plurality of drugs. Each response of the responses is indicative of a patient response to at least one drug in the plurality of drugs. In some instances, the mathematical model is not specific to a particular drug. In other instances, the model may be originally specific to a particular drug and then adapted over time, using the methods described herein, to apply to a class of similar drugs. In other instances, a user may find that a drug-specific model can be used to predict concentrations or responses for other drugs, and the drug-specific model may be used for drug-agnostic predictions.

The method includes forecasting, using the selected mathematical model and based on the inputs, a plurality of predicted concentration time profiles. Each of the predicted concentration time profiles may be indicative of a response of the patient to any drug in the plurality of drugs via a particular route of administration. Each predicted concentration time profile of the plurality of predicted concentration time profiles may correspond to a dosing regimen in a plurality of dosing regimens output by the system. Each dosing regimen may comprise at least one dose amount and/or a recommended schedule for administering the at least one dose amount to the patient. The method may include selecting from the plurality of dosing regimens a first dosing regimen for the plurality of drugs to achieve a treatment objective based on the target drug exposure or response level.

The method may include outputting the first dosing regimen for the plurality of drugs for the patient. At least one advantage of the method is that the model may access a broader range of clinical data compared to drug-specific models, because the model may be refined, for example, use measured data from administration of one drug in the set of drugs in order to provide better recommendations for dosing any of the drugs in the set of drugs.

In some implementations, the mathematical model is selected based on one or more criteria, such as the inputs, error associated with the models, or commonalities between each model and the plurality of drugs. In some implementations, the mathematical model is selected based on the drug data. If the model is selected based on the drug data, the selection step may involve comparing the parameters or covariates of the model to the drug data. This step may involve maximizing the number of similar parameters between the model and the set or class of drugs. Each model in the database may be associated with an error or confidence interval indicative of past performance of the model, and the model may be selected based on the error or confidence interval. The model may be selected based on the amount of data or information available for or applicable to the model. It would be advantageous for the selected model to use the broadest range of information or data available, so that the model can be refined to make more accurate predictions and to provide recommendations that are more likely to achieve the target. For example, the concentration or response data and/or the type of concentration or response data may be analyzed to select a model that is capable of incorporating that type of data into the model or capable of incorporating at least some or a maximum of the data. In some implementations, selection is performed by an optimization function. In lieu of or in addition to the previously discussed methods of selection, Bayesian methods be used to select the model. In some implementations, a plurality of models are compared to each other, and a model that best represents the patient or the inputs may be selected.

In some implementations, the drug data excludes information identifying the specific drug belonging to the plurality of drugs. The drug data may comprise one or more available dosage units corresponding to one or more drugs in the plurality of drugs. In such cases, the dose amount may be configured to be a multiple of the available dosage unit. In some implementations, the method further includes receiving additional drug data indicative of an updated route of administration for the specific drug or one or more drugs in the plurality of drugs. The system may update the mathematical model based on the updated route of administration for the specific drug or one or more drugs in the plurality of drugs. The updated mathematical model can then be used to calculate at least one updated dosing regimen to reach the treatment objective for the patient. The at least one updated dosing regimen may be outputted for the patient.

In some implementations, the model is a pharmacokinetic model. The model may alternatively be a pharmacodynamic model. The model may describe both pharmacokinetics and pharmacodynamics. Pharmacokinetic or pharmacodynamic components of the model may indicate concentration time profiles of the plurality of drugs. A pharmacokinetic component of the model may be based on clearance parameters representative of inflow and outflow of the drug(s) in the patient's body. A pharmacodynamic component of the model may be based on synthesis and degradation rates of a pharmacodynamic marker indicative of an individual response of the patient to the plurality of drugs. In some implementations, wherein the model comprises both a pharmacokinetic component and a pharmacodynamic component, the components are interrelated. For example, the clearance of the pharmacokinetic component may be a function of the pharmacodynamic response, and/or vice versa. The model may employ Bayesian methods, such as Bayesian forecasting to predict patient response or concentration time profiles for one or more dosing regimens.

In some implementations, the method further includes receiving additional patient data indicative of a second response of the patient to administration of the specific drug according to the first dosing regimen or a modified version of the first dosing regimen. The additional patient data may comprise additional concentration data indicative of one or more concentration levels of the specific drug in one or more samples obtained from the patient. The model can then be updated based on the second response of the patient to administration of the specific drug according to the first dosing regimen or the modified version of the first dosing regimen. At least one updated dosing regimen can be calculated, using the updated mathematical model, to reach the treatment objective for the patient. The at least one updated dosing regimen can be outputted for the patient.

The route of administration may be at least one of: subcutaneous, intravenous, oral, intramuscular, intrathecal, sublingual, buccal, rectal, vaginal, ocular, nasal, inhalation, nebulization, cutaneous, or transdermal. The plurality of drugs may be one of: monoclonal antibodies and/or antibody constructs, cytokines, drugs used for enzyme replacement therapy, aminoglycoside antibiotics, and chemotherapeutic agents that cause white cell decreases. In some implementations, each drug in the plurality of drugs shares a similar chemical structure. Each drug in the plurality of drugs may share a similar mechanism of action. In some implementations, the plurality of drugs are used to treat an inflammatory disease, for example, inflammatory bowel disease (IBD), rheumatoid arthritis, ankylosing spondylitis, psoriatic arthritis, psoriasis, asthma, and multiple sclerosis. The specific drug may be infliximab or adalimumab. The drug data may include dosing strength for the specific drug and/or an indicator representative of whether the specific drug is fully human or chimeric or fragmented. The system may receive as input patient data indicative of a patient disease to treat.

In some implementations, the received inputs include physiological data indicative of one or more measurements of at least one physiological parameter of the patient. The at least one physiological parameter of the patient may include at least one of: markers of inflammation, an albumin measurement, an indicator of drug clearance, a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score, a disease activity score (DAS), a Sharp score, and demographic information. The mathematical model may be selected from a set of mathematical models in order to best fit the received physiological data.

In some implementations, the method includes generating for display a patient-specific concentration time profile indicative of the patient response to the plurality of drugs in response to the first dosing regimen and an indication of at least some of the concentration data and the additional concentration data. An indication of the target drug exposure or response level may be generated for display. In some implementations, the system receives historical data indicative of a response of the patient to a second drug, other than the specific drug. The computational model may account for the historical data in order to generate predictions of concentration time profiles of the plurality of drugs in the patient. At least one advantage provided herein relates to the model being capable of applying a broader range of data than is typical, such as data gathered from one or more patients that were administered one or more drugs, in order to generate predictions for patient response to a class of drugs including drugs similar to the one or more drugs previously administered.

Another aspect relates to a method for generating a patient-specific medication dosing regimen using a computerized medication dosing regimen recommendation system without having previously measured concentration data. The method may be implemented on any of the systems described herein. The method involves receiving inputs into a processor of the system. The inputs may include drug data indicative of plurality of drugs and a route of administration. The drugs in the plurality of drugs may be expected to exhibit similar pharmacokinetic effects, similar pharmacodynamic effects, or both. The inputs may include initial drug concentration input data representative of an initial comparative concentration data point. A target drug exposure or response level is included in the inputs. The method further includes selecting a mathematical model representative of responses by a plurality of patients to one or more drugs in the plurality of drugs. Each response may be indicative of a patient response to at least one drug in the plurality of drugs. The mathematical model may be configured to be not specific to a particular drug.

The method includes using the selected model and the inputs to forecast a plurality of predicted concentration time profiles indicative of a response of the patient to any drug in the plurality of drugs via a route of administration. Each predicted concentration time profile may correspond to a dosing regimen in a plurality of dosing regimens. Each dosing regimen may comprise at least one dose amount and/or a recommended schedule for administering the at least one dose amount to the patient. The method includes selecting a first dosing regimen for the plurality of drugs forecasted to achieve a treatment objective based on the target drug exposure or response level. The first dosing regimen may be outputted for the patient. At least one advantage of the aspect results from the model providing predicted concentration time profiles and dosing regimens configured to meet the treatment objective using only the initial comparative concentration data point, without having measured concentration data from the specific patient.

In some implementations, the initial comparative concentration data point is indicative of a concentration level of a specific drug of the plurality of drugs in a sample obtained from the patient. Alternatively, the initial comparative concentration data point is calculated based on physiological parameters of the patient, such that direct concentration measurement from the patient is not required. The physiological parameters of the patient may include at least one of: markers of inflammation, an albumin measurement, an indicator of drug clearance, a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score, a disease activity score (DAS), a Sharp score, and demographic information. The drug data may include available dosing strength for the specific drug and/or an indicator representative of whether the specific drug is fully human, humanized, chimeric, or fragmented.

Another aspect relates to a system comprising a controller configured to perform the methods of the previous aspects. The system may comprise an interface connection to an electronic medical record (EMR) database. The interface may be operable by software of firmware configured to retrieve one or more indicators of patient information for a patient via the interface connection. An appropriate medicament for administration to the patient may be determined using the retrieved patient information. This aspect provides at least one advantage in that a physician may input a patient name, and the EMR for the patient will feed drug data and/or physiological data into the system automatically, in order to perform an initial forecast. This aspect may be particularly useful when recommending dose regimens for a relatively new drug, because the new drug may have structural similarities and similar identified covariates to other drugs, allowing a drug-agnostic model to be used by simply adjusting for the new drug particularities such as dosing strength to generate a dosing recommendation. For example, the new drug may only have clinical data from FDA trials. This clinical trial data can be supplied to drug-agnostic model system and normalized to other drugs in a class or group of similar drugs. Without much knowledge of the new drug, particularly knowledge that is used to dose ad hoc, physicians may use the system and methods provided herein to find proper doses for a new drug based on historical data of similar drugs.

Another aspect relates to a pharmaceutical formulation comprising a first dose amount of a specific drug. The first dose amount may be determined by the methods described herein. For example, the first dose amount of the drug in the pharmaceutical formulation is determined by a system to meet a treatment objective based on an inputted target exposure or response level of the drug, after having forecasted concentration time profiles for a plurality of drugs including the specific drug. The specific drug and each drug of the plurality of drug may have similar pharmacokinetics, pharmacodynamics, and/or structural characteristics. The pharmaceutical formulation may include a modified first dose, determined by the system using an updated mathematical model, the updated mathematical model taking into account concentration or response data indicative of a response by the patient to a dose of one or more drugs in the plurality of drugs.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 is a flowchart describing a method for determining a patient-specific dosing regimen for a set of drugs, according to an illustrative implementation;

FIG. 2 is a block diagram of a system for performing the methods described herein, according to an illustrative implementation;

FIGS. 3A and 3B are example graphs depicting simulated concentration-time profiles indicating a predicted patient response to a plurality of drugs for a given dosing regimen to meet a target level, based on a drug-agnostic model, according to an illustrative implementation;

FIG. 4 describes a process for developing a drug-agnostic model for a set of drugs, according to an illustrative implementation;

FIG. 5 is a flowchart showing a process for modifying adaptive dosing systems, according to an illustrative implementation;

FIG. 6 shows a system diagram of a computer network for adaptive dosing systems, according to an illustrative implementation; and

FIGS. 7A and 7B are example displays of a user interface on a clinical portal that provide several recommended dosing regimens, according to an illustrative implementation.

### DETAILED DESCRIPTION

The systems and methods described herein determine a patient-specific pharmaceutical dosing regimen for a patient, using a computerized pharmaceutical dosing regimen recommendation system applied to a class or other grouping of drugs. The systems and methods described herein provide population models capable of being used for a class or set of drugs. In particular, the systems and methods herein can be configured to develop patient-specific dosing regimens based on data indicative of the pharmacokinetic (“PK”) parameters and pharmacodynamic (“PD”) parameters of a plurality of drugs. Examples of such drugs are included in Table 1. The following definitions are used in Table 1: “iv” is intravenous; “sc” is subcutaneous; “RA” is rheumatoid arthritis; “AS” is ankylosing spondylitis; “UC” is ulcerative colitis; “CD” is Crohn's disease; “IBD” is inflammatory bowel disease, which may include ulcerative colitis and/or Crohn's disease; “PSO” is psoriasis; “PSA” is psoriatic arthritis; and “MS” is multiple sclerosis. In some implementations, the systems described herein may not be specific to a particular drug but instead apply to a class, or other subset or grouping of drugs (e.g., drugs that are expected to have a similar PK/PD, drugs known to be candidates of treating a particular condition, or other point of similarity). The development and application of drug-agnostic models allow greater utility

TABLE 1
Examples of drugs that may be grouped for developing patient-
specific dosing regimens, wherein mechanism, source, indication,
and route of administration may be factors in a model.

| Generic Name | Brand Name | Mechanism | Source | Indication | Route |
|---|---|---|---|---|---|
| infliximab | Remicade | anti-tnf | chimeric | RA AS IBD PSO PSA | iv |
| adalimumab | Humira | anti-tnf | human | RA AS IBD PSO PSA | sc |
| vedolizumab | Entyvio | anti α4β7 integrin | chimeric | IBD | iv |
| ustekinumab | Stelara | anti-IL-12 and IL-23 | human | IBD PSO PSA | iv sc |
| golimumab | Simponi | anti-tnf | human | RA AS PSA UC | sc |
| abatacept | Orencia | anti-CD80/CD86 | fusion | RA | iv sc |
| rituximab | Rituxan | anti-CD20 B Cell | chimeric | RA | iv |
| ixekizumab | Taltz | anti-IL-17 | chimeric | PSO PSA | sc |
| certolizumab | Cimzia | anti-tnf | fab | RA AS PSA UC | sc |
| pegol | | | fragment | | |
| etanercept | Enberel | anti-tnf | fusion | RA PSA | sc |
| dupilumab | Dupixant | anti IL4 receptor | human | PSO atopic dermatitis | sc |
| | | | | asthma | |
| tocilizumab | Actemra | anti-IL6 | chimeric | RA | iv sc |
| alemtuzumab | Lemtrada | anti-CD52 | chimeric | MS | iv |
| secukinumab | Cosentyx | anti-IL-17 | human | PSO PSA AS | sc |
| guselkumab | Tremfya | anti-IL-23 | human | PSO | sc |
| reslizumab | Cinqair | anti-IL-5 | chimeric | asthma | iv |
| mepolizumab | Nucala | anti-IL-5 | chimeric | asthma | sc |
| omalizumab | Xolair | IgE | chimeric | asthma | sc |
| benralizumab | Fasenra | CD125 | chimeric | asthma | sc |
| sarilumab | Kevzara | anti IL6 receptor | human | RA | sc |
| risankizumab | Skyrizi | anti-IL-23 | chimeric | PSO | sc |
| tildrakizumab | Ilumya | anti-IL-23 | chimeric | PSO | sc |
| ocrelizumab | Ocrevus | anti-CD20 | chimeric | MS | sc |
| natalizumab | Tysabri | anti α4 integrin | chimeric | MS | iv |
| canakinumab | Ilaris | anti-IL-1β | human | cryopyrin-associated | sc |
| | | | | periodic syndromes | |

for a single model. Rather than implementing multiple models, where each model corresponds to a single drug in a class, a drug-agnostic model is applicable to all drugs within the class and may accordingly be applied to a wider range of patients than single-drug models and across a range of administration routes.

For example, the models described herein may include a pharmacokinetic drug-agnostic model capable of being used for all biologics used in the treatment of inflammatory diseases. Such a model can be used to propose and/or test dose regimens for fully human monoclonal antibodies (mAbs), chimeric mAbs, humanized mAbs, fusion proteins, and mAb fragments (i.e., a range of drugs with differing pharmacokinetic properties but other similarities such as similar molecular weight and indication), such as those listed in Table 1, using the same model. The model may be used in a broad patient population, including inflammatory bowel disease, rheumatoid arthritis, psoriatic arthritis, psoriasis, multiple sclerosis, and other such diseases that arise from immune dysregulation. The development and application of drug-agnostic Bayesian models for agents in other broad drug sets (e.g., the aminoglycoside antibiotics, chemotherapeutic agents that cause low white cell counts, etc.) is similarly feasible. Drugs within the class may be administered through a variety of routes, such as subcutaneous, intravenous, oral, intramuscular, intrathecal, sublingual, buccal, rectal, vaginal, ocular, nasal, inhalation, nebulization, cutaneous, or transdermal. Drug-agnostic models may account for route of administration by taking the route of administration as a variable input to the system, allowing greater flexibility for the model. A computational model, such as a Bayesian model may be used to determine dosing regimen recommendations. For example, each iteration of the model may include a calculation or a determination of a recommended dosing regimen. When additional data is made available (such as physiological parameter data or drug concentration data obtained from the patient), another iteration of the model may be performed to determine an updated recommended dosing regimen based on the additional data. This process may be repeated any number of times to reflect any new data that describes the patient.

As used herein, a “dosing regimen” includes at least one dose amount of a drug or class of drugs and a recommended schedule for administering the at least one dose amount of the drug to a patient. The dose amount may be a multiple of an available dosage unit for the drug. For example, the available dosage unit could be one pill or a suitable fraction of a pill that results when it is easily split, such as half a pill. In some implementations, the dose amount may be an integer multiple of the available dosage unit for the drug. For example, the available dosage unit could be a 10 mg injection or a capsule that cannot be split. For some routes of administration (e.g., IV and subcutaneous), any portion of the dose strength can be administered. The recommended schedule includes a recommended time for administering a next dose of the drug to the patient, such that a predicted concentration time profile of the drug in the patient in response to the first pharmaceutical dosing regimen is at or above the target drug exposure or response level (e.g., a target drug concentration trough level) at the recommended time.

Because the drug-agnostic model applies to a set of drugs, rather than only a single drug, the model may retain patient-specific information when a patient is treated with multiple drugs within the set of drugs. For example, the set of drugs may include infliximab, vedolizumab, adalimumab, and other anti-inflammatory biologics. If a patient is treated with one drug (e.g. infliximab), then later treated with another drug (e.g. vedolizumab), the model may retain all patient-specific data (drug concentration measurements, clearance rates, weight measurements, etc.) from the patient's treatment on infliximab when determining an appropriate dosing regimen once the patient is being treated with the new drug. Retaining patient-specific data allows the drug-agnostic model to accurately anticipate the patient's ability to process a drug and thereby provide more suitable, patient-specific dosing regimens when a patient changes drug therapy. Because the drug-agnostic model(s) can fit to a broad range of data, with multiple routes of application, and a broad range of diseases, a model should learn about the drug and the individual patient (e.g., via Bayesian learning). Such drug-agnostic pharmacokinetic models, for example, represent a novel application of traditional population pharmacokinetic modeling. The ability to develop such a drug-agnostic pharmacokinetic (PK) model can be predicated on one or more of several factors, including: 1) a common universal structural PK model for all agents in a specific class, 2) similar effects of patient factors on the PK parameters, and 3) similar indications. Thus, the development and application of drug-agnostic Bayesian models for agents in other broad drug classes (e.g. the aminoglycoside antibiotics) is similarly feasible and will allow greater utility of a single drug-agnostic model rather than implementation of multiple models for each drug in a class. The development of a particular “class” for drug-agnostic modeling purposes is a novel means of classification or categorization for drugs.

Similarly, a drug-agnostic model can be constructed for drug classes that exhibit a commonality for the pharmacodynamic effect (the measured response of a drug). For example, many chemotherapeutic agents cause neutropenia or low white cell counts. This is a delayed response, with the lowest white cell counts generally occurring 7 to 9 days after the chemotherapy is administered. The impact of each drug on the duration, and nadir of white counts may differ but the underlying relationship between drug exposure and decrease in white cell count is structurally similar, allowing a practical drug-agnostic pharmacodynamic model to be developed for the class of chemotherapeutic agents that cause white cell decreases.

In some implementations, the drug-agnostic model describes pharmacokinetics and pharmacodynamics. The model includes a PK component and a PD component, which may be separate within the model, or they may be interrelated. For example, the PK and PD components may be interrelated such that the effects of PK on PD and PD on PK are included in the model. The PK component can include a PK clearance parameter and the PD component includes a PD response parameter. The interrelation between the PK and PD components may be reflected by PK clearance parameter being a function of the PD response, or vice versa. One or more differential equations can be used to describe the patient response and clearances of the drugs in the patient. A PD component of the model may comprise a first differential equation and a PK component of the model comprises a second differential equation. The first differential equation may represent PD response by the patient, and the second differential equation may represent PK clearance by the patient. The first or second differential equation may include PD response and/or PK clearance.

The systems and methods described herein may output a recommended dosing regimen for a class of drugs without identifying a specific drug to administer to the patient. As used herein, a “dosing regimen” may include a dose amount of a drug and a recommended schedule for administering the dose amount to a patient. The recommended schedule includes a recommended time for administering a next dose of the drug to the patient, to achieve a predicted concentration time profile of the drug in the patient in response to the first pharmaceutical dosing regimen that is at or above a target, for example, a drug concentration trough level, at the recommended time.

A class of drugs indicates a group of drugs larger than one, which exhibit at least one similar PK or PD effect, or share a common mechanism of action, or some other similarity. For example, a similar PK effect may be clearances within a specific range. A similar effect may be a measured concentration within a specific range, for example, bioavailability, absorption, a white cell count, blood concentration level, or any of the biomarkers/measurements discussed herein. The specific range may be within a tenfold difference, i.e. values of 0.1 to 1 may be considered similar. The specific range may be specified by a user on the system interface. The drugs may be grouped into a class by the disease they treat, such as general inflammatory disease, or more particularly inflammatory bowel disease (IBD), ulcerative colitis, Crohn's disease, rheumatoid arthritis, ankylosing spondylitis, psoriatic arthritis, psoriasis, asthma, or multiple sclerosis. Drug class may also be based on drug structure. For example, a class may include monoclonal antibodies (mAbs), chimeric mAbs, fully human mAbs, humanized mAbs, fusion proteins, and/or mAb fragments. Classes of medications may include anti-inflammatory compounds, chemotherapeutics, corticosteroids, immunomodulators, antibiotics or biologic therapies, or any other suitable group. Drug classes may be further determined by patient population, i.e., pediatrics, geriatrics. Drug classes may also be determined by a user based on other criteria, and members of that class (or other group) may be electronically designated in a data base as being part of that class (or group). That database is accessible to systems and methods disclosed herein, for use in determining a class (or other group) based dosing regimen. A drug class or group may include variations of the same drug, such as the same drug with different routes of administration or different manufacturers. This feature may be particularly useful if a physician needs to compare generic and brand-name drugs which vary in price, availability, indication, and/or route. Many of the examples described herein are in relation to the pharmaceutical infliximab. However, the implementations described herein may apply to immunosuppressive, anti-inflammatory, antibiotic, anti-microbial, chemotherapy, anti-coagulant, pro-coagulant, anti-depressant, anti-psychotics, psychostimulants, anti-diabetic, anti-convulsant, analgesic, or any other suitable treatment.

Many of the implementations described herein relate to the treatment of IBD, such as ulcerative colitis or Crohn's disease. Although there is no standard treatment regimen for IBD, the following groups of drugs can be used to treat IBD patients: anti-inflammatory compounds, corticosteroids, immunomodulators, antibiotics or biologic therapies. One recently developed treatment includes biologic therapies (e.g., monoclonal antibodies (mAbs) such as infliximab), which target and bind to an inflammatory protein called tumor necrosis factor (TNF), rendering it inactive. In some instances, a combination of anti-TNF agents, such as infliximab, can be combined with one or more immunomodulatory agents, such as thiopurines. Such combination therapies may effectively lower elimination rates (thereby increasing drug concentration levels in a patient's blood) and reduce formation of anti-drug antibodies. The biggest challenge in treating a patient with IBD is ensuring that the patient receives adequate exposure to the treatment. The body presents several routes of “clearance” for the drugs. For example, a patient's metabolism may break down mAbs by proteolysis (breaking down of proteins), by cellular uptake, and by additional atypical clearance mechanisms associated with IBD. For example, due to the nature of the disease, patients with conditions such as focal segmental glomerulosclerosis (FSGS) often suffer from excessive losses of the drug into the urinary and or gastrointestinal tracts. Moreover, in severe IBD, mAbs are sometimes lost in feces through ulcerated and denuded mucosa, creating an additional route of clearance. Overall, IBD patients are estimated to have an infliximab elimination rate that is 40% to 50% higher than other inflammatory diseases, making IBD especially difficult to treat. The systems and methods described herein may also develop dosing regimens to treat rheumatoid arthritis, psoriatic arthritis, ankylosing spondylitis, plaque psoriasis, low levels of clotting factor VIII, hemophilia, schizophrenia, bipolar disorder, depression, bipolar disorder, infectious diseases, cancer, seizures, transplants, or any other suitable affliction.

Inputs into the system may be used to update and refine the model for a specific patient taking a specific drug. Inputs into the systems described herein may include concentration data, physiological data, and a target response. The inputs to the model generally include concentration data, physiological data, and a target response. As discussed above, the concentration data is indicative of one or more concentration levels of a drug in one or more samples obtained from the patient, such as blood, blood plasma, urine, hair, saliva, or any other suitable patient sample. The concentration data may reflect a measurement of the concentration level of the drug itself in the patient sample, or of another analyte in the patient sample that is indicative of the amount of drug in the patient's body. The drug may be part of a treatment plan to treat a patient with a particular health condition, such as a disease or disorder like inflammatory bowel disease (IBD, including ulcerative colitis and Crohn's disease), rheumatoid arthritis, psoriatic arthritis, ankylosing spondylitis, plaque psoriasis, or any other suitable affliction. Drugs used to treat such health conditions may include monoclonal antibodies (mAbs), such as infliximab or adalimumab. While many of the examples described herein are with reference to using infliximab to treat IBD, it will be understood that the systems and methods of the present disclosure are applicable to any drug or treatment that loses its effectiveness over time in a measurable way, and may be used to treat any number of diseases, including any inflammatory disease, such as IBD.

Inputs to the system may also include other drug information, such as disease to be treated, class of drugs, route of administration, dose strength available, preferred dosing amount (e.g., 100 mg vial, 50 mg tablets, etc.), and whether the specific drug is fully human or not (e.g., chimeric). The drug information may be used to determine the available treatment options for a patient, the selected model, and the model parameters. For example, patients treated for IBD often have a higher clearance rate than those without IBD, and a drug dosing regimen for a treatment with IBD must be adjusted accordingly. The preferred dosing amount may alter a dosing regimen before the regimen is recommended for a patient. For example, if a drug is only available in 100 mg vials, the recommended dose amount may be rounded to the nearest 100 mg increment. In some implementations, the drug information excludes information identifying the drug currently used to treat the patient. For example, the drug data may be generic to a drug class. The physiological data is generally indicative of one or more measurements of at least one physiological parameter of the patient. This may include at least one of: medical record information, markers of inflammation, an indicator of drug elimination such as an albumin measurement or a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score, a disease activity score (DAS), a Sharp/van der Heijde score, and demographic information.

The target response may be selected by a physician based on his/her assessment of the patient's tolerance and response to drug therapy. In an example, the target response includes a target drug concentration level of a drug in a sample obtained from the patient (such as a concentration maximum, minimum, or exposure window), and may be used to determine when a patient should receive a next dose and an amount of that next dose. The target drug concentration level may include a target drug concentration trough level; a target drug concentration maximum; a target drug area under the concentration time curve (AUC); both a target drug concentration maximum and trough; a target pharmacodynamic endpoint such as blood pressure or clot time; or any suitable metric of drug exposure. The target may be decided by a physician based on the drug data and/or concentration or response. In some implementations, a target may be automatically determined by the system in order to result in a therapeutic response in the patient. The system may evaluate a plurality of targets inputted in order to determine one or more targets that result in a therapeutic response in the patient. The inputs described above (e.g., the concentration data, the physiological data, drug information, and the target response) are used by the systems and methods of the present disclosure to personalize a dosing regimen recommendation for a patient.

Based on the received inputs, the systems and methods described herein set one or more parameter values for a computational model (such as any of the model parameters described in U.S. patent application Ser. No. 15/094,379 (the '379 application), published as U.S. Patent Application Publication No. 2016/0300037, filed Apr. 8, 2016, and entitled “Systems and Methods for Patient-Specific Dosing”, which is hereby incorporated by reference in its entirety) that generates predictions of concentration time profiles of the drug in the patient. In some implementations, the computational model is a Bayesian model. For example, the computational model may take into account historical and/or present patient data to develop a patient-specific targeted dosing regimen. As discussed in the '379 application, the computational model may comprise a pharmacokinetic component indicative of a concentration time profile of the drug, and a pharmacodynamic component based on synthesis and degradation rates of a pharmacodynamic marker indicative of the patient's individual response to the drug. The computational model may be selected from a set of computational models that best fits the received physiological data. For example, if a patient is a 45 year old man, the system may select a computational model specific to men between the ages of 30 and 50 years of age. This computational model can be individualized to a specific patient by accounting for patient-specific measurements (such as the additional concentration data and additional physiological parameter data described herein).

Patient-specific drug dosing regimens may be provided as a function of mathematical models (e.g., pharmacokinetic and/or pharmacodynamic models) that are updated to account for observed patient responses. This is described in detail in the '379 application, which is incorporated herein by reference in its entirety. In particular, a specific patient's observed response to an initial dosing regimen is used to adjust the dosing regimen. The patient's observed response (e.g., an observed drug concentration in the patient's blood) is used in conjunction with the mathematical model and patient-specific characteristics to account for between-subject-variability (BSV) that cannot be accounted for by the mathematical model alone. The observed responses of the specific patient can be used to refine the models and related forecasts, to effectively personalize the models so that they may be used to forecast expected responses to proposed dosing regimens more accurately for a specific patient. In this manner, observed patient-specific response data is effectively used as “feedback” to adapt a generic model describing typical patient response to a patient-specific model capable of accurately forecasting a patient-specific response, such that a patient-specific dosing regimen can be predicted, proposed and/or evaluated on a patient-specific basis. Using the observed response data to personalize the models allows the models to be modified to account for BSV that is not accounted for in previous mathematical models, which described only typical responses for a patient population, or a “typical for covariates” response for a typical patient having certain characteristics accounted for as covariates in the model.

The systems and methods may rely on Bayesian analysis. For example, Bayesian analysis may be used to determine an appropriate dose needed to achieve a desirable result, such as maintaining a drug's concentration in the patient's blood near a particular level. Bayesian analysis may involve Bayesian forecasting and Bayesian updating. These Bayesian techniques may be used to develop a model that is a function not only of patient-specific characteristics accounted for in the model as covariate patient factors, but also observed patient-specific responses that are not accounted for within the models themselves, and that reflect between-subject-variability (BSV) that distinguishes the specific patient from the typical patient reflected by the model. In this manner, the present disclosure accounts for variability between individual patients that is unexplained and/or unaccounted for by traditional mathematical models (e.g., patient response that would not have been predicted based solely on the dose regimen and patient factors). Further, the present disclosure allows patient factors accounted for by typical models, such as weight, age, race, laboratory test results, etc., to be treated as continuous functions rather than as categorical (cut off) values. By doing this, the model is adapted to a specific patient, such that patient-specific forecasting and analysis can be performed, to predict, propose and/or evaluate dosing regimens that are personalized for a specific patient.

Notably, the present disclosure may be used to not only retroactively assess a dosing regimen previously administered to the patient, but also to prospectively assess a proposed dosing regimen before administering the proposed dosing regimen to the patient, or to identify dosing regimens (administered dose, dose interval, and route of administration) for the patient that will achieve the desired outcome. Bayesian forecasting process may be used to test various dosing regimens for the patient as a function of the patient's specific characteristics accounted for as patient factor covariates within the models, and the mathematical model. This forecasting involves evaluating dosing regimens based on predicted responses for a typical patient with the patient-specific characteristics. Generally, Bayesian forecasting involves using mathematical model parameters to forecast the likely response that a specific patient will exhibit with various dosing regimens. Notably, forecasting allows for determination of a likely patient response to a proposed dosing regimen before actual administration of a proposed dosing regimen. Accordingly, the forecasting can be used to test multiple different proposed dosing regimens (e.g., varying dose amount, dose interval and/or route of administration) to determine how each dosing regimen would likely impact the patient, as predicted by the patient-specific factors and/or data in the model/composite model. The forecasts may be compared to create a set of satisfactory or best dosing regimens for achieving the treatment objective or target exposure or concentration level. For example, the target may involve maintenance of a trough blood concentration level above a therapeutic threshold.

In some implementations, the recommended dosing regimen is provided with a confidence interval that indicates a likelihood that the particular dosing regimen will be therapeutically effective for the patient. In particular, the confidence interval of the projected response or concentration from the individual data may be assessed based on the complexity of the model and the amount of individual data (PK and/or PD data). In particular, the confidence interval may reflect the possible error in the individual predictions from the models. Initially, when no individual measurements have been taken from the patient, the model's predictions have an error associated with them that is approximately equal to the unexplained variability in the PK and the PD models. However, as individual measurements are taken and introduced into these models, the error (or equivalently, the confidence interval) decreases before ultimately approaching the assay error, which may correspond to a measurement error. Moreover, the confidence intervals may be provided to a clinical portal, to give a medical professional a sense for the amount of error remaining in the model predictions.

A Bayesian update process may be used to update the model based on the patient's response to the dosing regimen. Generally, Bayesian updating involves a Bayesian inference, which is a method in which Bayes' rule is used to update the probability estimate for a hypothesis as additional evidence is obtained. Bayesian updating is especially important in the dynamic analysis of data collected over time (sequentially). The method as applied here uses models that describe not only the time course of exposure and/or response, but also include terms describing the unexplained (random) variability of exposure and response. The result of Bayesian updating is a set of parameters conditional to the observed data. The process involves sampling parameters from a prior distribution (e.g., the underlying models) and calculating the expected responses based on the underlying models. For each underlying model, the difference between the model expectation and the observed data is compared. This difference is referred to as the “objective function.” The parameters are then adjusted based on the objective function, and the new parameters are tested against the observed data by comparing the difference between the new model expectation and the observed data. This process runs iteratively until the objective function is minimized, suggesting that the parameters that minimize the objective function best describe the current data. In some implementations, a random function may be used to interject some variation to ensure that a drug-agnostic minimum of the objective function has been obtained.

In some instances, the systems and methods of the present disclosure determine a dosing regimen recommendation based on how a patient responded to previous treatment methods from within or beyond the class (or other group) of drugs. As an example, when a patient does not respond to a treatment involving one drug (e.g., a previously administered drug), physicians sometimes switch the patient to a different drug (e.g., a currently administered drug); the drugs may be related to one another (e.g., from the same class of sharing a common mechanism of action). Lack of response to treatment is sometimes referred to as treatment “failure”, which can sometimes occur in patients with high elimination rates. In these patients, the drug is often eliminated from the body before the full beneficial effects of the drug are realized by the body. Generally speaking, an IBD patient with a high elimination rate for one drug (previously-administered) may be expected to also have a high elimination rate for another similar drug (yet to be administered) because the mechanisms of clearance for similar drugs are generally similar as well. In this manner, the elimination rate (as reflected by the measured drug concentration levels) of the previously administered drug may inform the elimination rate of the drug to be administered. The systems and methods described herein may exploit this correlation to use historical patient data involving one drug (e.g., a previously administered drug) in determining a recommended dosing regimen for another drug (e.g., a drug currently used to treat the patient). For example, a doctor may prescribe adalimumab for a patient. The patient may have a higher-than-average rate of clearance for adalimumab. If the patient fails therapy on adalimumab, the doctor may then attempt therapy with infliximab. The systems and methods described herein then take the higher-than-average clearance of adalimumab into account when calculating dosing regimens for infliximab, because the patient is likely to clear infliximab at a higher-than-average rate as well owing to the fact that both adalimumab and infliximab are mAbs with similar clearance mechanisms.

The systems and methods described herein determine and provide recommended dosing regimens using an iterative approach. In an example, an initial dosing regimen (determined based on the patient's available information and the physician's experience) is administered to a patient. Data indicative of the patient's response or reaction to the initial dosing regimen, such as the patient's physiological and/or concentration data, is provided to the system as feedback on the initial dosing regimen. Then, all, some, or none of that data is used as inputs to a computational model that calculates an updated dosing regimen that is provided to the physician as a recommendation. The physician may choose to administer the dosing regimen exactly as recommended, or the physician may choose to slightly alter the recommended dosing regimen before administering it. For example, the recommended dosing regimen may include a specific dosing interval (e.g., 4 weeks) and a specific dose amount (e.g., 1.9 vials). The physician may select to alter the regimen to accommodate the patient's schedule (e.g., if the patient can only return for another dose at 4 weeks and 1 day), to round up to a particular number of vials (e.g. to 2 vials), or both. This iterative approach is described in detail below.

Using the computational model and the set parameters described above, the systems and methods describe herein determine a first pharmaceutical dosing regimen for the patient. The first pharmaceutical dosing regimen comprises at least one dose amount of the drug and a recommended schedule for administering the at least one dose amount of the drug to the patient. The recommended schedule includes a recommended time for administering a next dose of the drug to the patient, such that a predicted concentration time profile or pharmacodynamic marker profile of the drug in the patient in response to the first pharmaceutical dosing regimen is at or above the target drug exposure level at the recommended time. In some implementations, the dosing regimen may be displayed (e.g., through a user interface). In some implementations, a physician may receive or view (e.g., through a user interface) the first dosing regimen. The physician may decide to alter the dosing regimen before it is administered to a patient. Once the physician has decided on a course of treatment, doses may be administered to the patient and additional data (indicative of how the patient is responding to the doses) is received.

In some implementations, after a start of administration of a dosing regimen based at least in part on the first pharmaceutical dosing regimen to the patient, the systems and methods described herein receive additional concentration data and additional physiological data obtained from the patient. A medical professional may choose to administer the dosing regimen to the patient exactly as recommended by the system. However, the medical professional may also choose to alter the first pharmaceutical dosing regimen prior to administration of the drug. For example, the medical professional may choose to round the dose amount up or down, may choose to alter the dosing times to better fit patient and doctor schedules, may make any suitable alteration to the first pharmaceutical dosing regimen, or any suitable combination thereof. For example, the first pharmaceutical dosing regimen may stipulate a dose of 100 mg on April 20 but the physician may decide to instruct administration of 90 mg on April 22. Details of the administered dose(s) can then be input into the system. The system may then receive additional data (e.g., concentration and physiological parameter data) indicative of how the patient is responding to the administered dose(s).

Often, when a patient is early in treatment, there is not much patient data. In particular, historical data indicative of how the patient has responded or reacted to different doses of the medication is typically unavailable. In this case, when there is little patient data to rely on, the systems and methods of the present disclosure may limit the number of concentration data points considered in an iteration of the model to just a single data point, which may be the most recent data point. The most recent data point may be the sole concentration data input to the model because that data point most accurately reflects the current status of the patient, and allows the systems and methods of the present disclosure to heavily weight that data point in the determination of a recommended dosing regimen. In this manner, the inputs to the model are determined based on whether an administered period of treatment of the first pharmaceutical dosing regimen is greater than a proportion of a total length of time of the first pharmaceutical dosing regimen (e.g., whether the patient is early in treatment or not).

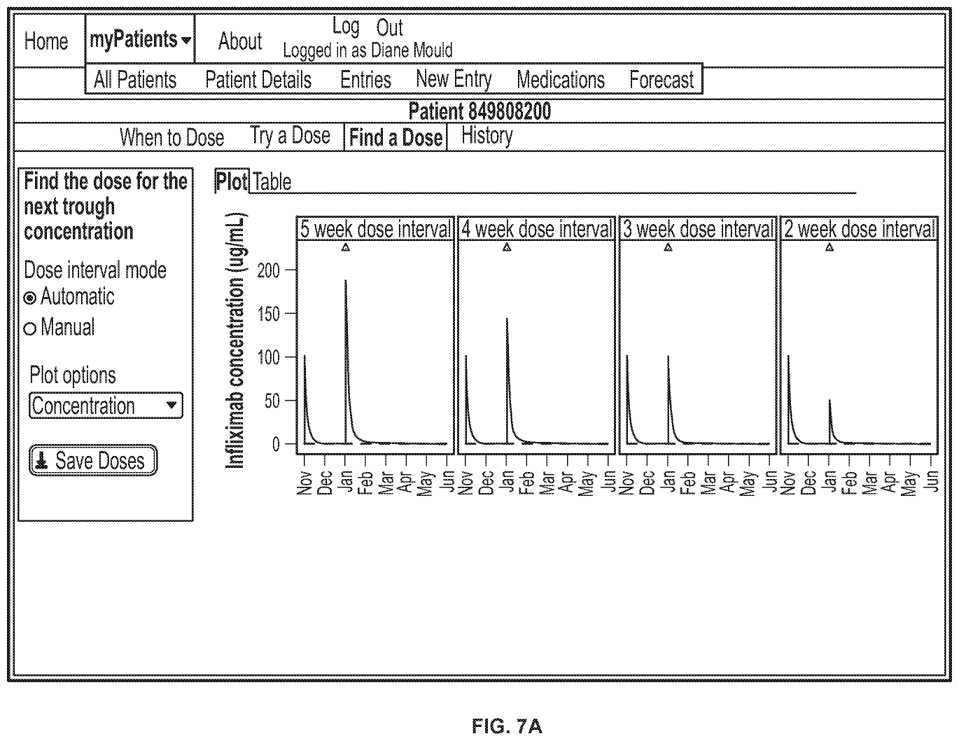

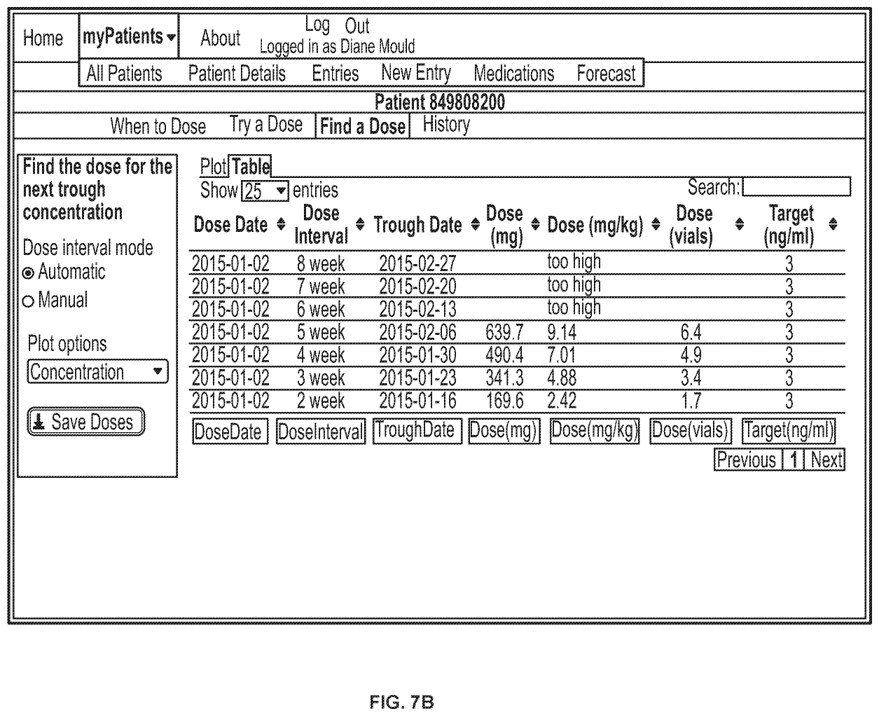

The systems and methods described herein may display information to a user. The system may display information through a user interface that physician or other user can interact with. For example, the user interface may display a dosing regimen (e.g., as shown in FIGS. 7A and 7B and described below) or may display the concentration and/or physiological parameter data. In some implementations, a user can toggle or otherwise choose which data to view on the user interface. In some implementations, the predicted concentration time profile of the drug in the patient in response to the first pharmaceutical dosing regimen, as generated by the computational model, is displayed. An indication of at least some of the concentration data and the additional concentration data, the physiological data and the additional physiological data, the target drug exposure level, and the recommended time as a point where the predicted concentration time profile of the drug intersects with the target drug exposure level may also be displayed. For example, a physician may use a graphical user interface to input target responses for a patient, input patient data (e.g., physiological and concentration data), and view predicted concentration time profiles for the patient in response to different dosing regimens.

In some implementations, inputs, such as the inputs described above, are retrieved from an electronic medical record. For example, a doctor may input a patient's name into the dosing system, which then may communicate with an electronic database system (stored locally in the dosing system or remotely) holding a plurality of patient information including health information for the instant patient. The electronic database system's health information may include, for instance, a current drug the patient is taking. The dosing system may then receive information from the electronic database system about the patient's health and/or drug information. In some examples, the drug information (such as drug class, routes of administration, minimum dosing amount, or any other suitable information) are retrieved from a separate database or server once a drug name is identified. Such systems may be advantageous, for example, in reducing the time necessary for a clinician to input information manually and reducing the chance of input errors by automatically pulling information from these databases. Such EMR applications may be activated or deactivated in a given system, depending on the context and need.

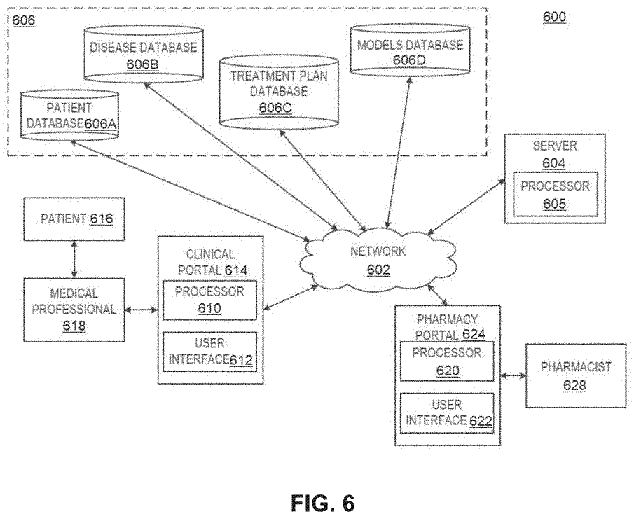

The systems and methods described herein may be used to predict patient drug clearance for a class (or other group) of drugs. Such models may be standardized to account for differences between drugs within the group of drugs. In some implementations, the model is created by collecting parameter values from a set of published models corresponding to a class of drugs. The parameter values may be collected in a lookup table. The parameter values may be converted to “standardized values” so they can be compared or pooled within the drug-agnostic model. This allows the system to simulate PK characteristics for patient populations for a published model with covariate effects as published, and for patient populations for an extended published model with all measured and presumed covariate effects. Standardized parameters may include body weight, albumin, ADA negative, presence of immune-suppressants, CRP, glucose, human or chimeric, non-IBD disease, sex, non-linear clearance, and CL. The lookup table may be used to normalize parameters to allow preliminary estimates from a drug-agnostic model. The lookup table may be manipulated by a user through a user interface, and may be stored in model database 606D of FIG. 6. The lookup table may be structured so that subsets of the table can be sent to simulation functions in a program so that each drug can be easily simulated in a variety of scenarios. Simulated concentrations from normalized parameters for each drug in the group of drugs may be compared and analyzed with respect to pooled data for that group of drugs, so as to fit the simulated concentration data to the pooled data. The drug-agnostic model for the group of drugs provides a set of parameters that applies to or is representative of all drugs in that group.

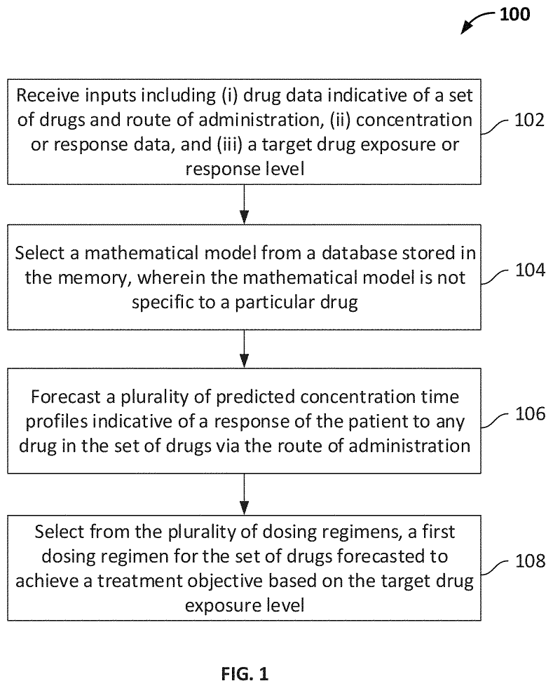

FIG. 1 shows a process 100 implemented by a system or a method for providing dosing regimens for a plurality of drugs (also referred to as a set of drugs), according to an illustrative implementation. In step 102, inputs to the system are received. Inputs include drug data indicative of a set of drugs and route of administration, concentration or response data, and a target drug exposure or response level. In some implementations, the drugs in the set of drugs are expected to exhibit similar PK behavior, similar PD behavior, or both. In some implementations, the drug data includes indications for each drug such as intended patient population or disease. In some implementations, the drug data includes information about the structure or function of the drugs, such as the mechanism of action. Information about the structure of the drug may include, for example, if one or more drugs in the set of drugs are antibodies, whether each antibody is chimeric, fragmented, humanized, or fully human. It is possible that the drug data may be supplied without identifying a specific drug, enabling a physician to get simulated results, e.g., predicted concentration time profiles, clearances, and/or dosing regimens, without identifying or constraining the results to a specific drug. The concentration or response data may be indicative of a concentration or response level of a specific drug of the set of drugs in a sample obtained from the patient. The concentration or response data may be further indicative of a concentration or response level of a specific drug administered using a specific route of administration in a sample obtained from the patient. Patient-specific covariates or measurements, such as body weight, albumin, ADA negative, presence of immune-suppressants, CRP levels, glucose levels, concomitant disease(s), disease status, and sex, may also be included as inputs to the system.

In step 104, a mathematical model is selected from a database stored in memory (e.g., model database 606D in FIG. 6). The database is by a processor and stores a plurality of mathematical models. The mathematical model is representative of responses by a plurality of patients to a plurality of drugs in the set of drugs, wherein each response of the responses is indicative of a patient response to at least one drug in the set of drugs, and wherein the mathematical model is not specific to a particular drug. The mathematical model may be selected based on one or more of the inputs to the system.

In some implementations, the mathematical model is selected based on the drug data. If the model is selected based on the drug data, the selection step may involve comparing the parameters or covariates of the model to the drug data. This step may involve maximizing the number of similar parameters between the model and the set or class of drugs. Each model in the database may be associated with an error or confidence interval indicative of past performance of the model, and the model may be selected based on the error or confidence interval. The model may be selected based on the amount of data or information available for or applicable to the model. It would be advantageous for the selected model to use the broadest range of information or data available, so that the model can be refined to make more accurate predictions and to provide recommendations that are more likely to achieve the target. For example, the concentration or response data and/or the type of concentration or response data may be analyzed to select a model that is capable of incorporating that type of data into the model or capable of incorporating at least some or a maximum of the data. In some implementations, selection may be performed by an optimization function. In lieu of or in addition to the previously discussed methods of selection, Bayesian methods be used to select the model. In some implementations, a plurality of models are compared to each other, and a model that best represents the patient or the inputs may be selected. In certain implementations, the model is a Bayesian model, which may be capable of performing Bayesian analysis such as Bayesian forecasting.

In certain implementations, parameters may be set for the computational model that generates predictions of concentration time profiles of a drug in a patient. The parameters are set based on the received inputs of step 102. In some implementations, the computational model is a Bayesian model. In some implementations, the computational model comprises a pharmacokinetic component indicative of a concentration time profile of the drug, and a pharmacodynamic component based on synthesis and degradation rates of a pharmacodynamic marker indicative of an individual response of the patient to the drug. In some implementations, process 300 comprises an additional, optional step where the computational model is selected from a set of computational models that best fits the received physiological data. In some implementations, the computational model accounts for historical data indicative of a response of the patient to a historical drug in order to generate predictions of concentration time profiles of the drug in the patient. In some implementations, the historical drug may belong to the same class of drugs as the current drug. In some implementations, the historical drug may belong to a different class of drugs from the current drug.

In step 106, a plurality of predicted concentration time profiles are forecasted. The predicated concentration time profiles are forecasted using the selected mathematical model and based on the concentration data and the route of administration. Each predicted concentration time profile of the plurality of predicted concentration time profiles corresponds to a dosing regimen in a plurality of dosing regimens. Each dosing regimen of the plurality of dosing regimens may comprise at least one dose amount, and a recommended schedule for administering the at least one dose amount to the patient. In some implementations, the recommended schedule is determined, using the model, to maintain the target concentration or exposure or response level based on the at least one dose amount or on the concentration or response data. Step 106 may involve Bayesian forecasting.

In step 108, a first dosing regimen for the set of drugs is selected. The first dosing regimen is forecasted to achieve a treatment objective based on the target drug exposure or response level. The first dosing regimen may be output from the system. In some implementations, the first dosing regimen is output for display on a user device. In some implementations, a plurality of first dosing regimens are output. In some implementations, a physician may choose the first dosing regimen from the plurality of first dosing regimens. Selection of the first dosing regimen may be determined by predefined criteria or by scores associated with each dosing regimen in the plurality of dosing regimens. For example, a user may select the dosing regimen that best meets a treatment objective or best fits a target concentration or concentration time profile. The score may be a percentage calculated by the system representative of how closely the predicted concentration time profiles for a particular dosing regimen fits the target. The score may be a p-value as is known in the state of the art. The score may indicate residual error. For example, the score may be the mean residual error associated with simulation using the particular dosing regimen or may be presented as a confidence interval. These scores may be output with the corresponding dosing regimen.

In an example, upon viewing the selected/recommended first dosing regimen over a user interface, a medical professional may select to administer the first dosing regimen as recommended, or the medical professional may select to slightly alter the recommended dosing regimen, such as by changing one or more dates or times in the recommended schedule to accommodate the patient's or professional's schedule, and/or by changing the dosage amount (e.g., by rounding up to the nearest integer of vials, for example). A dosing regimen (e.g., either the recommended dosing regimen, or a modified version of the recommended dosing regimen) is administered by the medical professional, for example through oral medication; intravenous, intramuscular, intrathecal, or subcutaneous injection; insertion rectally or vaginally; infusion; topical, nasal, sublingual, or buccal application; inhalation or nebulization; the ocular route or the otic route; or any other suitable administration route. The dose amount may be a multiple of an available dosage unit for the drug. For example, the available dosage unit could be one pill or a suitable fraction of a pill that results when it is easily split, such as half a pill. In some implementations, the dose amount may be an integer multiple of the available dosage unit for the drug. For example, the available dosage unit could be a 10 mg injection or a capsule that cannot be split. For some routes of administration (e.g., IV and subcutaneous) any portion of the dose strength can be administered.

The professional (or another user of the system) may provide data indicative of the actual administered dosing regimen to the system. In an example, if the administered dosing regimen is the same as the recommended dosing regimen, the user may simply select a button on the user interface indicating the recommended dosing regimen was selected to be administered. Alternatively, if the administered dosing regimen is different from the recommended dosing regimen, the user provides data indicative of the administered dosing regimen to the system. After a start of administration of the dosing regimen to the patient (which may be the same as or a modified version of the recommended dosing regimen provided by the model), the system receives additional data indicative of observed responses of the patient to the dosing regimen. In particular, the system may receive additional concentration data, additional physiological data, or both. The system receives additional concentration data, which is concatenated with the concentration data. For example, the additional concentration data may be representative of the patient's response to the dosing regimen administered, and may be a single data point or multiple data points. Additionally or alternatively, the system receives additional physiological parameter data, which is concatenated with the concentration data. For example, the additional physiological parameter data may be representative of the patient's response to the dosing regimen administered, and may be a single data point, or multiple data points in a vector or a matrix form. The system might receive additional concentration data without receiving additional physiological parameter data. Alternatively, the system may receive additional physiological parameter data without receiving additional concentration data. In another example, the system may receive both additional concentration data and additional physiological data.

The method may comprise additional steps. In some implementations, the additional steps include receiving additional drug data indicative of an updated route of administration for the specific drug; updating the mathematical model, based on the update route of administration for the specific drug; calculating, based on the updated mathematical model, at least one updated dosing regimen to reach the treatment objective for the patient; and outputting the at least one updated dosing regimen for the patient. In such implementations, these additional steps allow a physician to vary the route of administration, while analyzing the entire set of drugs, in order to identify one or more dosing regimens that provide the best care to the patient, i.e., best meets the treatment objective. In some implementations, the additional steps include receiving additional patient data indicative of a response of the patient to administration of the specific drug according to the first dosing regimen or a modified version of the first dosing regimen, the additional patient data comprising additional concentration data indicative of one or more concentration levels of the specific drug in one or more samples obtained from the patient; updating the mathematical model, based on the second response of the patient to administration of the specific drug according to the first dosing regimen or the modified version of the first dosing regimen; calculating, based on the updated mathematical model, at least one updated dosing regimen to reach the treatment objective for the patient; and outputting the at least one updated dosing regimen for the patient. The updating step may involve Bayesian updating.

In some implementations, the system also receives as inputs certain drug constraints such as dose strength available, preferred dose strength, available dosage unit, manufacturing schedule, vial strength, or price. These constraints may be incorporated in the above-described steps. For example, the first dosing regimen may be generated to include a dose amount that is a multiple of the available dosage unit. The dosing schedule in a dosing regimen may be configured to be a multiple of the manufacturing schedule. The system may be configured to determine a dose that best meets the treatment objective at the lowest cost to the patient based on the input drug costs. The route of administration may be at least one of: subcutaneous, intravenous, oral, intramuscular, intrathecal, sublingual, buccal, rectal, vaginal, ocular, nasal, inhalation, nebulization, cutaneous, or transdermal. The plurality of drugs may be one of: monoclonal antibodies and/or antibody constructs, cytokines, drugs used for enzyme replacement therapy, aminoglycoside antibiotics, and chemotherapeutic agents that cause white cell decreases.

In some implementations, each drug in the plurality of drugs shares a similar chemical structure. Each drug in the plurality of drugs may share a similar mechanism of action. In some implementations, the plurality of drugs are used to treat an inflammatory disease, for example, inflammatory bowel disease (IBD), rheumatoid arthritis, ankylosing spondylitis, psoriatic arthritis, psoriasis, asthma, and multiple sclerosis. In certain implementations, the specific drug is one of the drugs listed in Table 1. The drug data may include dosing strength for the specific drug and/or an indicator representative of whether the specific drug is fully human or chimeric or fragmented, in order to, for example, satisfy parameters that may be included in the mathematical model and describe similarities or differences between the drugs in the plurality of drugs. The system may receive, as an input, patient data indicative of a patient disease to treat, in order to, for example, classify drugs that are applicable to the patient disease or retrieve information relating to the patient disease that is used in the model or various steps.

In some implementations, the received inputs include physiological data indicative of one or more measurements of at least one physiological parameter of the patient. The at least one physiological parameter of the patient may include at least one of: markers of inflammation, an albumin measurement, an indicator of drug clearance, a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score, a disease activity score (DAS), a Sharp score, and demographic information. The mathematical model may be selected from a set of mathematical models in order to best fit the received physiological data.

In some implementations, the method includes generating for display a patient-specific concentration time profile indicative of the patient response to the plurality of drugs in response to the first dosing regimen and an indication of at least some of the concentration data and the additional concentration data. An indication of the target drug exposure or resopnse level may be generated for display. In some implementations, the system receives historical data indicative of a response of the patient to a second drug, other than the specific drug. The computational model may account for the historical data in order to generate predictions of concentration time profiles of the plurality of drugs in the patient. Thus the model may be capable of using a broader range of data, such as data gathered from administration of a drug other than the specific drug, in order to generate predictions for patient response to the plurality of drugs. The drug-agnostic model may supplement the lack of drug specificity with observed patient response data or historical drug data, for example, clinical trial data, in order to predict patient responses to dosing regimens and recommend optimized dosing regimens.

In some implementations, the system receives, as an input, initial drug concentration input data representative of an initial comparative concentration data point. In some cases, a user or physician may not have access to measured concentration or response data, and instead the system must estimate or calculate a first point based on physiological data. In some cases, the initial comparative concentration data point is indicative of a concentration level of a specific drug of the plurality of drugs in a sample obtained from the patient. In some cases, the initial comparative concentration data point is calculated based on physiological parameters of the patient. The first dosing regimen may be calculated based on forecasting of predicted concentration time profiles which are based on the selected mathematical model and the input data which is obtained from physiological parameters of the specific patient.

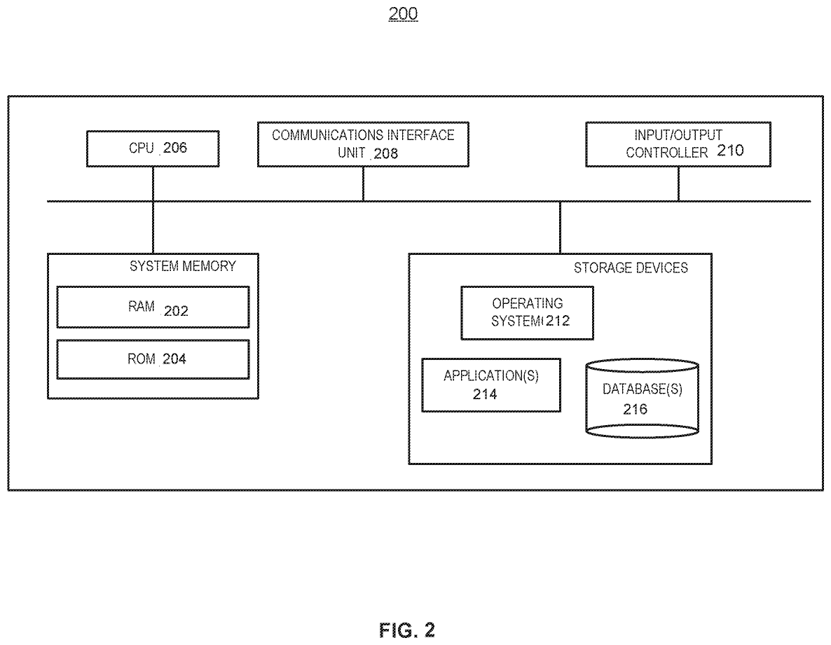

FIG. 2 is a block diagram of a computing device for performing any of the processes described herein. Each of the components of these systems may be implemented on one or more computing devices 200. In certain aspects, a plurality of the components of these systems may be included within one computing device 200. In certain implementations, a component and a storage device may be implemented across several computing devices 200.

The computing device 200 includes at least one communications interface unit, an input/output controller 210, system memory, and one or more data storage devices. The system memory includes at least one random access memory (RAM 202) and at least one read-only memory (ROM 204). All of these elements are in communication with a central processing unit (CPU 206) to facilitate the operation of the computing device 200. The computing device 200 may be configured in many different ways. For example, the computing device 200 may be a conventional standalone computer or alternatively, the functions of computing device 200 may be distributed across multiple computer systems and architectures. In FIG. 200, the computing device 200 is linked, via network or local network, to other servers or systems.

The computing device 200 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 208 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 206 includes a processor, such as one or more conventional microprocessors and one or more supplementary co-processors such as math co-processors for offloading workload from the CPU 206. The CPU 206 is in communication with the communications interface unit 208 and the input/output controller 210, through which the CPU 206 communicates with other devices such as other servers, user terminals, or devices. The communications interface unit 208 and the input/output controller 210 may include multiple communication channels for simultaneous communication with, for example, other processors, servers or client terminals.

The CPU 206 is also in communication with the data storage device. The data storage device may include an appropriate combination of magnetic, optical or semiconductor memory, and may include, for example, RAM 202, ROM 204, flash drive, an optical disc such as a compact disc or a hard disk or drive. The CPU 206 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 206 may be connected to the data storage device via the communications interface unit 208. The CPU 206 may be configured to perform one or more particular processing functions.

The data storage device may store, for example, (i) an operating system 212 for the computing device 200; (ii) one or more applications 214 (e.g., computer program code or a computer program product) adapted to direct the CPU 206 in accordance with the systems and methods described here, and particularly in accordance with the processes described in detail with regard to the CPU 206; or (iii) database(s) 216 adapted to store information that may be utilized to store information required by the program.

The operating system 212 and applications 214 may be stored, for example, in a compressed, an uncompiled and an encrypted format, and may include computer program code. The instructions of the program may be read into a main memory of the processor from a computer-readable medium other than the data storage device, such as from the ROM 204 or from the RAM 202. While execution of sequences of instructions in the program causes the CPU 206 to perform the process steps described herein, hard-wired circuitry may be used in place of, or in combination with, software instructions for implementation of the processes of the present invention. Thus, the systems and methods described are not limited to any specific combination of hardware and software.

Suitable computer program code may be provided for performing one or more functions described herein. The program also may include program elements such as an operating system 212, a database management system and “device drivers” that allow the processor to interface with computer peripheral devices (e.g., a video display, a keyboard, a computer mouse, etc.) via the input/output controller 210.

The term “computer-readable medium” as used herein refers to any non-transitory medium that provides or participates in providing instructions to the processor of the computing device 200 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 206 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 200 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

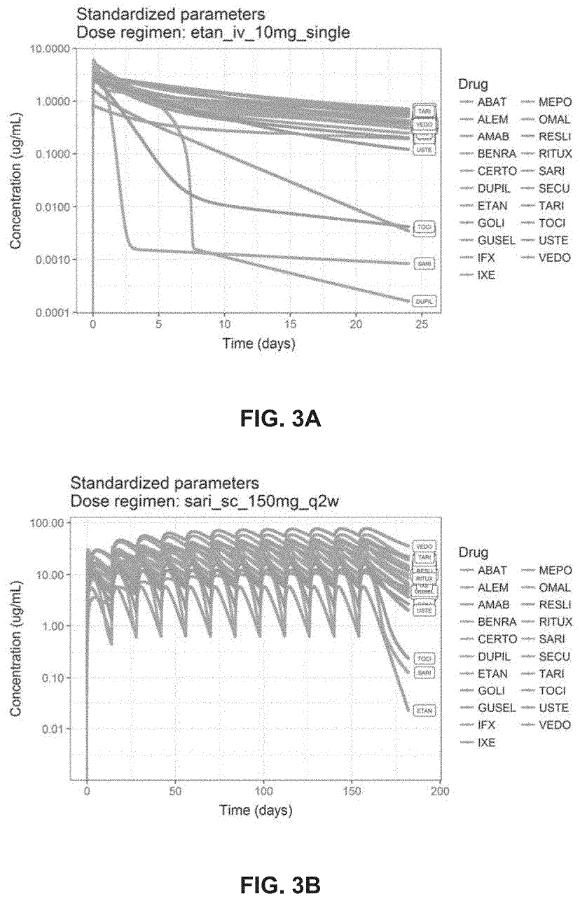

FIGS. 3A and 3B are graphs of predicted concentration time profiles for a specific patient in response to dosing regimens for each drug in a class of drugs. In each graph, concentration time profiles for the specific patient are simulated for a class of drugs using a drug-agnostic model, such that each profile corresponds to a drug in the class of drugs. The class of drugs simulated here includes various mAb's which have share some commonalities, as described in the foregoing. In this illustrative example, the dosing regimens are the standard dose and interval recommended for each drug in the package insert. In particular, FIG. 3A shows predicted concentration time profiles for the class of drugs. One of the applied standard dosing regimens is denoted above the chart, the regimen comprising 10 mg of etanercept (“ETAN”) administered intravenously in a single dosing. When concentrations are allowed to fall to very low (sub-therapeutic) values, for example, if the patient is not compliant with the dosing regimen, the profiles become significantly dissimilar, due to the differences between the drugs. These differences are reflected by the standardized parameters in the drug-agnostic model and may include certain aspects of pharmacokinetics and/or pharmacodynamics relating to the drugs.

FIG. 3B shows predicted concentration time profiles for the class of drugs. One of the applied standard dosing regimens is denoted above the chart, the regimen comprising 150 mg of sarilumab (“SARI”) administered subcutaneously biweekly. The profiles depicted in FIG. 3B are relatively similar, at least following similar trends and maintaining concentrations at higher values over time. For example, this simulation may represent a patient who is compliant with the dosing regimen. Accordingly, the differences between drugs, and general variability, can be minimized by managing the patient at reasonable drug concentrations. Only when dosing regimens are not followed, or when a dosing regimen is inadequate, does variability arise between drugs in the class of drugs.

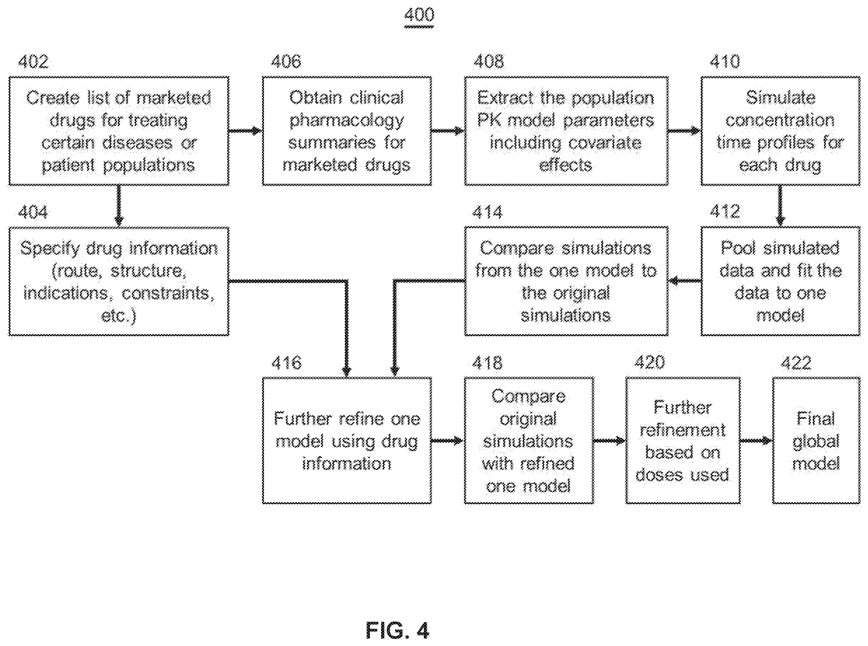

FIG. 4 depicts a flowchart which describes an exemplary method 400 of developing a drug-agnostic model, as described herein. Step 402 involves creating a list of marketed drugs for treating certain diseases or patient populations. These diseases or patient populations may be any of those described in the foregoing or any diseases or patient populations targeted by marketed drugs. For example, the list of marketed drugs may be sourced from literature or regulatory bodies, such as medical journals or the Food and Drug Administration (FDA). Table 1 may be an exemplary list of such drugs. Step 404 involves specifying drug information. Drug information, as similarly discussed with respect to step 102 of FIG. 1, may include route of administration, structure, indications, constraints, PK/PD parameters, or other data described herein. This information may be sourced from the same source as the list of drugs or may be entered by a user. Step 406 involves obtaining clinical pharmacology summaries for the marketed drugs. The pharmacology information is used in the drug-agnostic model to account for similarities or differences between the marketed drugs. Step 408 involves extracting the population PK model parameters. Step 410 involves simulating concentration time profiles for each drug. Step 412 involves pooling the simulated data and fitting the data to one model, i.e., the drug-agnostic model. Step 414 involves comparing the simulations from the one model to the original simulations. Step 416 involves further refining the one model using the drug information from step 404. Step 418 involves comparing the original simulations with the refined one model. Step 420 involves further refining the one model based on doses used, for example, based on the concentration or response data described in relation to step 102 of FIG. 1. In step 422, the final drug-agnostic model is provided.

In some implementations, in relation to step 408, the model is created by collecting parameter values for a set of published models corresponding to a class of drugs. It is to be understood that step 408 may alternatively or additionally involve extracting population PD model parameters. In implementations, where a PD model is desired, response data and response targets may be used in lieu of concentration data and concentration targets. The parameter values may be collected in a lookup table or the databases described below in FIG. 6. The lookup table may be manipulated by a user or automatically by the system as needed. The lookup table may be located anywhere in the system infrastructure, such as the systems described in FIGS. 2 and 6. The parameter values may be converted to “standardized values” so they can be compared or pooled within the drug-agnostic model. This allows the system, in step 410, to simulate PK and/or PD characteristics for patient populations for a published model with covariate effects as published, and for patient populations for an extended published model with all measured and presumed covariate effects. The lookup table may be used to normalize parameters to allow preliminary estimates from a drug-agnostic model. The normalization process may occur during steps 412 or 416. The lookup table may be structured so that subsets of the table can be sent to simulation functions in a program so that each drug can be easily simulated in a variety of scenarios, for example, during step 410.

As described in step 414, simulated concentrations from normalized parameters for each drug in the group of drugs may be compared and analyzed with respect to pooled data for that group of drugs, so as to fit the simulated concentration data to the pooled data. The drug-agnostic model for the group of drugs provides a set of parameters that applies to or is representative of all drugs in that group—this feature represents a significant technical contribution. Another technical effect of the drug-agnostic model is realized in step 420, where data collected from administered doses can be used to further refine the model. As the model is agnostic to a specific drug, the data from doses used can be from any drug in the class of drugs, and the model will accept that data and become further refined, for example, through Bayesian updating, because the drugs in the class of drugs share similarities reflected in the drug-agnostic model parameters.

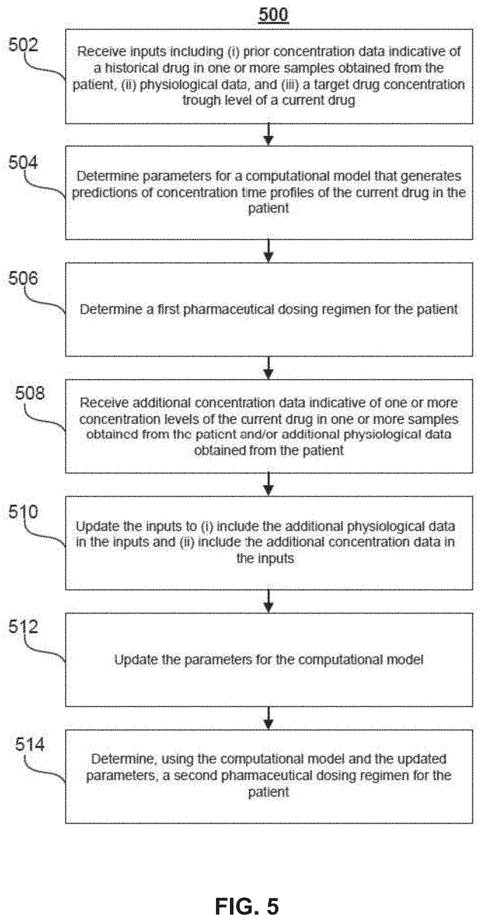

FIG. 5 shows a process 500 for using data from a patient's response to a prior treatment, to inform a recommended dosing regimen for a different treatment, according to an illustrative implementation. The process 500 can be performed using the computerized system 600 of FIG. 6, or any other suitable computerized system. In step 502, inputs to the system are received. The inputs include prior concentration data indicative of one or more prior concentration levels of a prior drug in one or more samples obtained from the patient, physiological data indicative of one or more measurements of at least one physiological parameter of the patient, and a target drug exposure or response level of a current drug. A patient may have failed therapy on the prior drug. Medical information (e.g., concentration data) indicating the patient's response to the prior drug may be useful in helping to determine how a patient will react to a current drug. In some implementations, the prior and current drug may belong to the same class or may have similar effects. For example, the prior and current drugs may be different monoclonal antibodies. If a patient's data indicates that the patient has a high elimination rate for the prior drug (e.g., the patient's body eliminates the prior drug from the patient's system at a higher rate than average or than is expected), then the patient is likely to have a similarly high elimination rate for the current drug. By retaining information from the prior drug, the systems and methods described herein may quickly determine an individualized dosing regimen for the current drug, thereby reducing the amount of time a patient is untreated if switching between drugs.

In step 504, parameters are set for a computational model that generates predictions of concentration time profiles of the current drug in a patient. The computational model may be a Bayesian model, a PK/PD model, any of the models described above in relation to FIGS. 1 and 4 and below in relation to FIG. 6 (e.g., those stored in models database 606D), or any suitable model. The parameters are set based on the received inputs of step 502. In some implementations, the computational model is a Bayesian model. In some implementations, the computational model comprises a pharmacokinetic component indicative of a concentration time profile of the drug, and a pharmacodynamic component based on synthesis and degradation rates of a pharmacodynamic marker indicative of an individual response of the patient to the drug. In some implementations, process 1500 comprises an additional, optional step where the computational model is selected from a set of computational models that best fits the received physiological data.

In step 506, a first pharmaceutical dosing regimen for the patient is determined using the computational model and the set parameters. The first pharmaceutical dosing regimen comprises (i) at least one dose amount of the drug and (ii) a recommended schedule for administering the at least one dose amount of the drug to the patient. The recommended schedule includes a recommended time for administering a next dose of the drug to the patient, such that a predicted concentration time profile of the drug in the patient in response to the first pharmaceutical dosing regimen is at or above the target drug concentration trough level at the recommended time. In step 508, additional concentration data corresponding to the current drug and/or additional physiological data are obtained from the patient. The additional concentration data and additional physiological data may result from administration of the first pharmaceutical dosing regimen to the patient. In some implementations, process 500 comprises additional steps providing for the display of various system inputs and outputs. In some implementations, process 1500 provides the predicted concentration time profile of the drug in the patient in response to the first pharmaceutical dosing regimen, as generated by the computational model, and an indication of at least some of the concentration data and the additional concentration data for display (e.g., through user interface 622 in FIG. 6). In some implementations, an indication of the target drug concentration trough level is provided for display (e.g., through user interface 622 in FIG. 6).

In step 510, the concentration data is updated to include both the concentration data of step 502 and the additional concentration data of step 508. In some implementations, at step 510, the original concentration data from step 502 is excluded, and the additional concentration data becomes the concentration data. In step 512, the parameters for the computational model are updated, based on the updated inputs, and in step 514, an iteration of the model is performed to determine a second pharmaceutical dosing regimen for the patient, based on the updated parameters.

FIG. 6 shows a block diagram of a computerized system 600 for implementing the systems and methods disclosed herein. In particular, the system 600 uses drug-specific mathematical models and observed patient-specific responses to treatment to predict, propose, modify and evaluate suitable drug treatment plans for a specific patient. The system 600 includes a server 604, a clinical portal 614, a pharmacy portal 624, and an electronic database 606, all connected over a network 602. The server 604 includes a processor 605, the clinical portal 614 includes a processor 610 and a user interface 612, and the pharmacy portal 624 includes a processor 620 and a user interface 622. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that is currently being processed. An illustrative computing device 200, which may be used to implement any of the processors and servers described herein, is described in detail above with reference to FIG. 2. As used herein, “user interface” includes, without limitation, any suitable combination of one or more input devices (e.g., keypads, touch screens, trackballs, voice recognition systems, etc.) and/or one or more output devices (e.g., visual displays, speakers, tactile displays, printing devices, etc.). As used herein, “portal” includes, without limitation, any suitable combination of one or more devices configured with hardware, firmware, and software to carry out one or more of the computerized techniques described herein. Examples of user devices that may implemental a portal include, without limitation, personal computers, laptops, and mobile devices (such as smartphones, blackberries, PDAs, tablet computers, etc.). For example, a portal may be implemented over a web browser or a mobile application installed on the user device. Only one server, one clinical portal 614, and one pharmacy portal 624 are shown in FIG. 6 to avoid complicating the drawing; the system 600 can support multiple servers and multiple clinical portals and pharmacy portals.

In FIG. 6, a patient 616 is examined by a medical professional 618, who has access to the clinical portal 614. The patient may be subject to a disease that has a known progression, and consults the medical professional 618. The medical professional 618 makes measurements from the patient 616 and records these measurements over the clinical portal 614. For example, the medical professional 618 may draw a sample of the blood of the patient 616, and may measure a concentration of a biomarker in the blood sample. In general, the medical professional 618 may make any suitable measurement of the patient 616, including lab results such as concentration measurements from the patient's blood, urine, saliva, or any other liquid sampled from the patient. The measurement may correspond to observations made by the medical professional 618 of the patient 616, including any symptoms exhibited by the patient 616. For example, the medical professional 618 may perform an examination of the patient to gather or measure physiological parameters or to determine drug concentrations in the patient. This involves identifying patient characteristics (physiological parameters and concentrations) that are reflected as patient factor covariates within the mathematical model that will be used to predict the patient's response to a drug treatment plan.

For example, if the model is constructed such that it describes a typical patient response as a function of weight and gender covariates, the patient's weight and gender characteristics would be identified. Any other characteristics may be identified that are shown to be predictive of response, and thus reflected as patient factor covariates, in the mathematical models. By way of example, such patient factor covariates may include markers of inflammation, an albumin measurement, an indicator of drug clearance, a measure of C-reactive protein (CRP), a measure of antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score and demographic information. Based on the patient's measurement data, the medical professional 618 may make an assessment of the patient's disease status, and may identify a drug or class of drugs suitable for administering to the patient 616 to treat the patient 616. The clinical portal 614 may then transmit the patient's measurements, the patient's disease status (as determined by the medical professional 618), and, optionally, an identifier of the drug over the network 602 to the server 604, which uses the received data to select one or more appropriate computational models from the models database 606.

The appropriate computational models are those that are determined to be capable of predicting the patient's response to the administration of the drug or class of drugs. The one or more selected computational models are used to determine a recommended set of planned dosages of the drug to administer to the patient, and the recommendation is transmitted back over the network 602 to the clinical portal 614 for viewing by the medical professional 618. Alternatively, the medical professional 618 may not be capable of assessing the patient's disease status or identify a drug, and either or both of these steps may be performed by the server 604. In this case, the server 604 receives the patient's measurement data, and correlates the patient's measurement data with the data of other patients in the patient database 606A. The server 604 may then identify other patients who exhibited similar symptoms or data as the patient 616 and determine the disease states, drugs used, and outcomes for the other patients. Based on the data from the other patients, the server 604 may identify the most common disease states and/or drugs used that resulted in the most favorable outcomes, and provide these results to the clinical portal 614 for the medical professional 618 to consider.

As is shown in FIG. 6, the database 606 includes a set of four databases including a patient database 606A, a disease database 606B, a treatment plan database 606C, and a models database 606D. These databases store respective data regarding patients and their data, diseases, drugs, dosage schedules, and computational models. In particular, the patient database 606A stores measurements, including physiological parameter data and concentration data, taken by or symptoms observed by the medical professional 618. The disease database 606B stores data regarding various diseases and possible symptoms often exhibited by patients infected with a disease. The treatment plan database 606C stores data regarding possible treatment plans, including drugs and dosage schedules for a set of patients. The set of patients may include a population with different characteristics, such as weight, height, age, sex, and race, for example.

The models database 606D stores data regarding a set of computational models that may be used to describe pharmacokinetic (PK), pharmacodynamic (PD), or both PK and PD changes to a body. Any suitable computational model may be stored in the models database 606D, such as in the form of a compiled library module, for example. In particular, a suitable mathematical model is a mathematical function (or set of functions) that describes the relationship between a dosing regimen and the observed patient exposure and/or observed patient response (collectively “response”) for a specific drug. Accordingly, the mathematical model describes response profiles for a population of patients. Generally, development of a mathematical model involves developing a mathematical function or equation that defines a curve that best “fits” or describes the observed clinical data. Typical models also describe the expected impact of specific patient characteristics on response, as well as quantify the amount of unexplained variability that cannot be accounted for solely by patient characteristics. In such models, patient characteristics are reflected as patient factor covariates within the mathematical model. Thus, the mathematical model is typically a mathematical function that describes underlying clinical data and the associated variability seen in the patient population. These mathematical functions include terms that describe the variation of an individual patient from the “average” or typical patient, allowing the model to describe or predict a variety of outcomes for a given dose and making the model not only a mathematical function, but also a statistical function, though the models and functions are referred to herein in a generic and non-limiting fashion as “mathematical” models and functions.

In particular, the output of the model corresponds to a dosing regimen or schedule that achieves an optimal target level for a physiological parameter of the patient 616. The model provides the optimal target level as a recommendation specifically designed for the patient 616, and has verified that the optimal target level is expected to produce an effective and therapeutic response in the patient 616. In the example shown, the concentration data corresponds to a concentration of a drug in the patient's blood. In some implementations, the drug may not be known—for example, the drug may be any drug in a class of drugs. The physiological parameter data may correspond to any number of measurements from a patient. When the drug is infliximab, for example, it may be desirable to measure the drug concentration (and predict the drug concentration using the model) and other measurable units (that may be predicted by the model), such as C reactive protein, endoscopic disease severity, and fecal calprotectin. Each measurable (e.g., the drug concentration, C reactive protein, endoscopic disease severity, and fecal calprotectin) may involve one or more models, such as PK or PD models. The interaction between PK and PD models may be particularly important for a drug like infliximab, in which patients with more severe disease clear the drug faster (modeled by higher clearance from a PK model, as is explained in detail below). One goal of the drug infliximab may be to normalize C reactive protein levels, lower fecal calprotectin levels, and achieve endoscopic remission.

In one example, the medical professional 618 may assess the likelihood that the patient 616 will exhibit a therapeutic response to a particular drug and dosing regimen. In particular, this likelihood may be low if several dosing regimens of the same drug have been administered to the patient, but no measurable response from the patient is detected. In this case, the medical professional 618 may determine that it is unlikely that the patient will response to further adjustments to the dose, and other drugs may be considered. Moreover, a confidence interval may be assessed for the predicted model results and the predicted response of the body to the presence of the drug. As data is collected from the patient 616, the confidence interval gets narrower, and is indicative of a more trustworthy result and recommendation. The systems and methods described herein may identify an individualized target level (e.g., target trough level), and may provide individualized dosing recommendations based on the individualized target level.

Often, the medical professional 618 may be a member or employee of a medical center. The same patient 616 may meet with multiple members of the same medical center in various roles. In this case, the clinical portal 614 may be configured to operate on multiple user devices. The medical center may have its own records for the particular patient. In some implementations, the present disclosure provides an interface between the computational models described herein and a medical center's records. For example, any medical professional 618, such as a doctor or a nurse, may be required to enter authentication information (such as a username and password) or scan an employee badge over the user interface 612 to log into the system provided by the clinical portal 614. Once logged in, each medical professional 618 may have a corresponding set of patient records that the professional is allowed to access. In some implementations, the patient 616 interacts with the clinical portal 614, which may have a patient-specific page or area for interaction with the patient 616. For example, the clinical portal 614 may be configured to monitor the patient's treatment schedule and send appointments and reminders to the patient 616. Moreover, one or more devices (such as smart mobile devices or sensors) may be used to monitor the patient's ongoing physiological data, and report the physiological data to the clinical portal 614 or directly to the server 604 over the network 602. The physiological data is then compared to expectations, and deviations from expectations are flagged. Monitoring the patient's data on a continual basis in this manner allows for possible early detection of deviations from expectations of the patient's response to a drug, and may indicate the need for modification of the dosing recommendation.

As described herein, the measurements from the patient 616 that are provided into the computational model may be determined from the medical professional 618, directly from devices monitoring the patient 616, or a combination of both. Because the computational model predicts a time progression of the disease and the drug, and their effects on the body, these measurements may be used to update the model parameters, so that the treatment plan (that is provided by the model) is refined and corrected to account for the patient's specific data. In some implementations, it is desirable to separate a patient's personal information from the patient's measurement data that is needed to run the computational model. In particular, the patient's personal information may be protected health information (PHI), and access to a person's PHI should be limited to authorized users. One way to protect a patient's PHI is to assign each patient to an anonymized code when the patient is registered with the server 604. The code may be manually entered by the medical professional 618 over the clinical portal 614, or may be entered using an automated but secure process. The server 604 may be only capable of identifying each patient according to the anonymized code, and may not have access to the patient's PHI. In particular the clinical portal 614 and the server 604 may exchange data regarding the patient 616 without identifying the patient 616 or revealing the patient's PHI. In some implementations, the clinical portal 614 is configured to communicate with the pharmacy portal 624 over the network 602. In particular, after a dosing regimen is selected to be administered to the patient 616, the medical professional 618 may provide an indication of the selected dosing regimen to the clinical portal 614 for transmitting the selected dosing regimen to the pharmacy portal 624. Upon receiving the dosing regimen, the pharmacy portal 624 may display the dosing regimen and an identifier of the medical professional 618 over the user interface 622, which interacts with the pharmacist 628 to fulfill the order.

As is shown in FIG. 6, the server 604 is a device (or set of devices) that is remote from the clinical portal 614. Depending on the computational power of the device that houses the clinical portal 614, the clinical portal 614 may simply be an interface that primarily transfers data between the medical professional 618 and the server 604. Alternatively, the clinical portal 614 may be configured to locally perform any or all of the steps described to be performed by the server 604, including but not limited to receiving patient symptom and measurement data, accessing any of the databases 606, running one or more computational models, and providing a recommendation for a dosage schedule based on the patient's specific symptom and measurement data. Moreover, while FIG. 6 depicts the patient database 606A, the disease database 606B, the treatment plan database 606C, and the models database 606D as being entities that are separate from the server 604, the clinical portal 614, or the pharmacy portal 624, one of ordinary skill in the art will understand that any or all of the databases 606 may be stored locally on any of the devices or portals described herein, without department from the scope of the present disclosure.

FIG. 7A depicts an example display screen (e.g., user interface 612 of FIG. 6) that displays predicted concentration time profiles for four different dosing regimens. In particular, each dosing regimen has a corresponding dose interval (5 weeks, 4 weeks, 3 weeks, and 2 weeks), where the dose amount decreases as dose interval decreases (as is indicated by the height of the second peak in each predicted concentration time profile). In this case, the user has selected to plot IFX concentration versus time, and to allow the computational models to run to identify recommended dosing regimens to maintain IFX concentrations above the critical trough value.

In FIG. 7B, the user has selected to display the results from the plot shown in FIG. 7A in a table form (e.g., on user interface 612 of FIG. 6). In particular, the example display screen in FIG. 7B lists the last dose date (Jan. 2, 2015), various dosing intervals, a trough date (corresponding to the first date after the dose date that the predicted concentration time profile falls to or at the critical trough value), the suggested dose (in mg), the normalized suggested dose (in mg/kg), the number of vials used for each dose, and the target concentration (in ng/ml). As is shown in FIG. 7B, a set of proposed dosing schedules is shown, where the dosing schedules have different dose intervals ranging from two to eight weeks. While some of the dosing schedules with longer dose intervals (six to eight weeks) are not recommended, four dosing schedules (with dose intervals of two to five weeks) are proposed with doses that increase as dose interval increases. In particular, when interacting with the display screen of FIG. 7B, the medical professional 618 may select a dosing regimen based on a specific goal. For example, the longer dose interval (e.g., five weeks) may be selected if it is desirable to administer doses to the patient 616 infrequently. Alternatively, since patients are often charged the price of a full vial, even when a partial vial is used, it may be desirable to use as much of the vials as possible. In this case, the four-week dosing regimen may be selected, since 4.9 vials are used for each dose, and leads to little wastage of the drug (e.g., only 0.1 vials per dosage). Alternatively, a shorter dose interval (e.g., two weeks) may be selected it if is desirable to administer doses to the patient 616 more frequently, or to charge the patient 616 for only two vials at a time.

It is to be understood that while various illustrative implementations have been described, the forgoing description is merely illustrative and does not limit the scope of the invention. While several examples have been provided in the present disclosure, it should be understood that the disclosed systems, components and methods of manufacture may be embodied in many other specific forms without departing from the scope of the present disclosure.

The examples disclosed can be implemented in combinations or sub-combinations with one or more other features described herein. A variety of apparatus, systems and methods may be implemented based on the disclosure and still fall within the scope of the invention. Also, the various features described or illustrated above may be combined or integrated in other systems or certain features may be omitted, or not implemented.

While various implementations of the present disclosure have been shown and described herein, it will be obvious to those skilled in the art that such implementations are provided by way of example only. Numerous variations, changes, and substitutions will now occur to those skilled in the art without departing from the disclosure. It should be understood that various alternatives to the implementations of the disclosure described herein may be employed in practicing the disclosure.

All references cited herein are incorporated by reference in their entirety and made part of this application.

1. A method for generating a patient-specific medication dosing regimen using a computerized medication dosing regimen recommendation system comprising a processor and a memory, the method comprising:
(a) receiving inputs into the processor of the system, the inputs including:
(i) drug data indicative of a plurality of drugs and associated routes of administration, wherein the drugs in the plurality of drugs are expected to exhibit similar pharmacokinetic (PK) behavior, similar pharmacodynamic (PD) behavior, or both,
(ii) concentration or response data indicative of a concentration or response level of a specific drug of the plurality of drugs in a sample obtained from the patient, and
(iii) a target drug exposure or response level for the patient;
(b) selecting a mathematical model from a database stored in the memory, the database being accessible by the processor and storing a plurality of mathematical models, wherein the selected mathematical model is representative of responses by a plurality of patients to a plurality of drugs in the plurality of drugs, wherein each response of the responses is indicative of a patient response to at least one drug in the plurality of drugs, and wherein the mathematical model is not specific to a particular drug;
(c) forecasting, using the selected mathematical model and based on the concentration data and the route of administration, a plurality of predicted concentration time profiles indicative of a response of the patient to any drug in the plurality of drugs via the route of administration, wherein each predicted concentration time profile of the plurality of predicted concentration time profiles corresponds to a dosing regimen in a plurality of dosing regimens, each dosing regimen of the plurality of dosing regimens comprising (i) at least one dose amount, and (ii) a recommended schedule for administering the at least one dose amount to the patient;
(d) selecting from the plurality of dosing regimens, a first dosing regimen for the plurality of drugs forecasted to achieve a treatment objective based on the target drug exposure or response level; and
(e) outputting the first dosing regimen for the plurality of drugs for the patient.

2. The method of claim 1, wherein the drug data excludes information identifying the specific drug belonging to the plurality of drugs.

3. The method of claim 1, the method further comprising:
receiving additional drug data indicative of an updated route of administration for the specific drug;
updating the mathematical model, based on the updated route of administration for the specific drug;
calculating, based on the updated mathematical model, at least one updated dosing regimen to reach the treatment objective for the patient; and
outputting the at least one updated dosing regimen for the patient.

4. The method of claim 3, wherein the drug data further comprises at least one of: one or more available dosage units corresponding to one or more drugs in the plurality of drugs, one or more available dosing strengths corresponding to one or more drugs in the plurality of drugs, or one or more indicators representative of whether one or more drugs in the plurality of drugs are fully human, and wherein the dose amount is a multiple of the available dosage unit.

5-6. (canceled)

7. The method of claim 3, the method further comprising:
receiving additional patient data indicative of a second response of the patient to administration of the specific drug or another drug of the plurality of drugs according to the first dosing regimen or a modified version of the first dosing regimen, the additional patient data comprising additional concentration data indicative of one or more concentration levels of the specific drug or another drug of the plurality of drugs in one or more samples obtained from the patient;
updating the mathematical model, based on the second response of the patient to administration of the specific drug or another drug of the plurality of drugs according to the first dosing regimen or the modified version of the first dosing regimen;
calculating, based on the updated mathematical model, at least one updated dosing regimen to reach the treatment objective for the patient; and
outputting the at least one updated dosing regimen for the patient.

8. The method of claim 7, wherein the route of administration is at least one of: subcutaneous, intravenous, oral, intramuscular, intrathecal, sublingual, buccal, rectal, vaginal, ocular, nasal, inhalation, nebulization, cutaneous, and transdermal, and wherein the plurality of drugs is one of: monoclonal antibodies and antibody constructs, cytokines, drugs used for enzyme replacement therapy, aminoglycoside antibiotics, and chemotherapeutic agents that cause white cell decreases.

9. (canceled)

10. The method of claim 8, wherein each drug in the plurality of drugs shares at least one of a similar chemical structure or a similar mechanism of action.

11-12. (canceled)

13. The method of claim 8, wherein the plurality of drugs is used to treat at least one of: an inflammatory disease, inflammatory bowel disease (IBD), rheumatoid arthritis, ankylosing spondylitis, psoriatic arthritis, psoriasis, asthma, and multiple sclerosis.

14-15. (canceled)

16. The method of claim 1, wherein the received inputs include at least one of: patient data indicative of a patient disease to treat or physiological data indicative of one or more measurements of at least one physiological parameter of the patient.

17. The method of claim 16, wherein the at least one physiological parameter of the patient includes at least one of: markers of inflammation, an albumin measurement, an indicator of drug clearance, a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PAST) score, a disease activity score (DAS), a Sharp score, and demographic information.

18. The method of claim 17, the method further comprising selecting the mathematical model from a set of mathematical models to best fit the received physiological data.

19. (canceled)

20. The method of claim 18, further comprising generating for display at least one of: (i) a patient-specific predicted concentration time profile indicative of the patient response to the plurality of drugs in response to the first dosing regimen, (ii) an indication of at least some of the concentration data and the additional concentration data, and (iii) an indication of the target drug exposure or response level.

21. (canceled)

22. The method of claim 20, wherein the model comprises a pharmacokinetic or pharmacodynamic component indicative of a concentration or response time profile of the plurality of drugs or based on synthesis and degradation rates of a pharmacodynamic marker indicative of an individual response of the patient to the plurality of drugs.

23. (canceled)

24. The method of claim 1, further comprising receiving historical data indicative of a response of the patient to a second drug, other than the specific drug, wherein the computational model accounts for the historical data in order to generate predictions of concentration time profiles of the plurality of drugs in the patient.

25-33. (canceled)

34. A system for generating a patient-specific medication dosing regimen, the system comprising:
a memory, and
at least one processor configured to:
(a) receive inputs into the processor of the system, the inputs including:
(i) drug data indicative of a plurality of drugs and associated routes of administration, wherein the drugs in the plurality of drugs are expected to exhibit similar pharmacokinetic (PK) behavior, similar pharmacodynamic (PD) behavior, or both,
(ii) concentration or response data indicative of a concentration or response level of a specific drug of the plurality of drugs in a sample obtained from the patient, and
(iii) a target drug exposure or response level for the patient;
(b) select a mathematical model from a database stored in the memory, the database being accessible by the at least one processor and storing a plurality of mathematical models, wherein the selected mathematical model is representative of responses by a plurality of patients to a plurality of drugs in the plurality of drugs, wherein each response of the responses is indicative of a patient response to at least one drug in the plurality of drugs, and wherein the mathematical model is not specific to a particular drug;
(c) forecast, using the selected mathematical model and based on the concentration data and the route of administration, a plurality of predicted concentration time profiles indicative of a response of the patient to any drug in the plurality of drugs via the route of administration, wherein each predicted concentration time profile of the plurality of predicted concentration time profiles corresponds to a dosing regimen in a plurality of dosing regimens, each dosing regimen of the plurality of dosing regimens comprising (i) at least one dose amount, and (ii) a recommended schedule for administering the at least one dose amount to the patient;
(d) select from the plurality of dosing regimens, a first dosing regimen for the plurality of drugs forecasted to achieve a treatment objective based on the target drug exposure or response level; and
(e) output the first dosing regimen for the plurality of drugs for the patient.

35. The system of claim 34, wherein the drug data excludes information identifying the specific drug belonging to the plurality of drugs.

36. The system of claim 34, the at least one processor being further configured to:
receive additional drug data indicative of an updated route of administration for the specific drug;
update the mathematical model, based on the updated route of administration for the specific drug;
calculate, based on the updated mathematical model, at least one updated dosing regimen to reach the treatment objective for the patient; and
output the at least one updated dosing regimen for the patient.

37. The system of claim 36, wherein the drug data further comprises at least one of: one or more available dosage units corresponding to one or more drugs in the plurality of drugs, one or more available dosing strengths corresponding to one or more drugs in the plurality of drugs, or one or more indicators representative of whether one or more drugs in the plurality of drugs are fully human, and wherein the dose amount is a multiple of the available dosage unit.

38-39. (canceled)

40. The system of claim 36, the at least one processor being further configured to:
receive additional patient data indicative of a second response of the patient to administration of the specific drug or another drug of the plurality of drugs according to the first dosing regimen or a modified version of the first dosing regimen, the additional patient data comprising additional concentration data indicative of one or more concentration levels of the specific drug or another drug of the plurality of drugs in one or more samples obtained from the patient;
update the mathematical model, based on the second response of the patient to administration of the specific drug or another drug of the plurality of drugs according to the first dosing regimen or the modified version of the first dosing regimen;
calculate, based on the updated mathematical model, at least one updated dosing regimen to reach the treatment objective for the patient; and
output the at least one updated dosing regimen for the patient.

41. The system of claim 40, wherein the route of administration is at least one of: subcutaneous, intravenous, oral, intramuscular, intrathecal, sublingual, buccal, rectal, vaginal, ocular, nasal, inhalation, nebulization, cutaneous, and transdermal, and wherein the plurality of drugs is one of: monoclonal antibodies and antibody constructs, cytokines, drugs used for enzyme replacement therapy, aminoglycoside antibiotics, and chemotherapeutic agents that cause white cell decreases.

42. (canceled)

43. The system of claim 41, wherein each drug in the plurality of drugs shares at least one of: a similar mechanism of action or a similar chemical structure.

44-45. (canceled)

46. The system of claim 43, wherein the plurality of drugs is used to treat at least one of: an inflammatory disease, inflammatory bowel disease (IBD), rheumatoid arthritis, ankylosing spondylitis, psoriatic arthritis, psoriasis, asthma, and multiple sclerosis.

47-48. (canceled)

49. The system of claim 34, wherein the received inputs include at least one of: patient data indicative of a patient disease to treat or physiological data indicative of one or more measurements of at least one physiological parameter of the patient.

50. The system of claim 49, wherein the at least one physiological parameter of the patient includes at least one of: markers of inflammation, an albumin measurement, an indicator of drug clearance, a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PAST) score, a disease activity score (DAS), a Sharp score, and demographic information.

51. The system of claim 50, the method further comprising selecting the mathematical model from a set of mathematical models to best fit the received physiological data.

52. (canceled)

53. The system of claim 51, wherein the at least one processor is further configured to generate for display at least one of: (i) a patient-specific predicted concentration time profile indicative of the patient response to the plurality of drugs in response to the first dosing regimen, (ii) an indication of at least some of the concentration data and the additional concentration data, and (iii) an indication of the target drug exposure or response level.

54. (canceled)

55. The system of claim 53, wherein the model comprises a pharmacokinetic or pharmacodynamic component indicative of a concentration time profile of the plurality of drugs or based on synthesis and degradation rates of a pharmacodynamic marker indicative of an individual response of the patient to the plurality of drugs.

56. (canceled)

57. The system of claim 34, the at least one processor is further configured to receive historical data indicative of a response of the patient to a second drug, other than the specific drug, wherein the computational model accounts for the historical data in order to generate predictions of concentration time profiles of the plurality of drugs in the patient.

58-59. (canceled)

60. The system of claim 34, wherein the system is a cloud-based computing system operated by the at least one processor.

61. The system of claim 60, wherein the cloud-based computing system comprises a network and at least one server, the network configured to link at least two selected from the group of: the at least one processor, the memory, the at least one server, and at least one user device.

62. The system of claim 60, further comprising an interface connection to an electronic medical record database, interface connection being operable by software or firmware configured to retrieve one or more indicators of patient information for a patient via the interface connection.

63-75. (canceled)
